SEQUENCE LISTING (38 sequences)

SEQ ID NO: 1 - protein, 90 aa, Unknown
  Description of Unknown HU family DNA-binding protein
MNKADLISAVAAEAGLSKVDAKKAVEAFVSTVTKALQEGDKVSLIGFGTFSVAERSARTGINPSTKATITIPAKKVTKFKPGAELADAIK

SEQ ID NO: 2 - DNA, 13 bp, Artificial Sequence
  Description of Artificial Sequence Synthetic oligonucleotide
cctgtctctccgc

SEQ ID NO: 3 - DNA, 10396 bp, Artificial Sequence
  Description of Artificial Sequence Synthetic polynucleotide
ggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatattcgtttaatatcataaataatttattttattttaaaatgcgcgggtgcaaaggtaagaggttttattttaactaccaaatgttttcggaagttttttcgcttttctttttctatcgtttctcagactctcttagcgaaagggaaagaaggtaaagaagaaaaacaaaacgccttttcttttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacaccatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaattaatgcggctgcaatttttttgggcggggccgcccaaaaaaatcctagcaccctgcagcagtactgcttgaccataagaacaaaaaaacttccgataaagtttggaagataaagctaaaagttcttatctttgcagtatgacgggaatgtaccccaggttttagagctagaaatagcaagttaaaataaggctagtccgttatcaacttgaaaaagtggcaccgagtcggtgctttttttgagatctgtcgactctagaggatccccgggtaccgagctcgaattcactggccgtcgttttacaacgtcgtgactgggaaaaccctggcgttacccaacttaatcgtacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtcctttttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcactggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgttctttacccgctgcttatctgcatcccatttggctacgtcaatccggtagcccgttgtaaactcgatacgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccggtaccgaggacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtgg

SEQ ID NO: 4 - DNA, 20 bp, Artificial Sequence
  Description of Artificial Sequence Synthetic oligonucleotide
aattgaggcatcggtccgaa

SEQ ID NO: 5 - DNA, 20 bp, Artificial Sequence
  Description of Artificial Sequence Synthetic oligonucleotide
atgacgggaatgtaccccag

SEQ ID NO: 6 - DNA, 20 bp, Artificial Sequence
  Description of Artificial Sequence Synthetic oligonucleotide
tgatggagaggtgcaagtag

SEQ ID NO: 7 - DNA, 82 bp, Artificial Sequence
  Description of Artificial Sequence Synthetic oligonucleotide
gttttagagctagaaatagcaagttaaaataaggctagtccgttatcaacttgaaaaagtggcaccgagtcggtgctttttt

SEQ ID NO: 8 - DNA, 20 bp, Artificial Sequence
  Description of Artificial Sequence Synthetic oligonucleotide
attcggcagtgaattccaga

SEQ ID NO: 9 - DNA, 9321 bp, Artificial Sequence
  Description of Artificial Sequence Synthetic polynucleotide
ctcgagtccccgaccgatgatttttaagtgactgattttgtgctgttttgggggtatattaagaataaaagaaatagaataagttaagtacttgatacacaatatagggcattttccatattggaaattctcattttccaatctagaaaataccgatttcctaatatgatactaaataggaaaataatatttcccttaatattgtttttatggaaaataatagtttactttgtggagaataatatttcccaaaaacatatcaaaatggaaaataaaaaagcagttaagttaaccgattttcaaaagaagcaagaaaatccttttatgaaaacaagctatagaggtattgaaaatcatgttgttaaaaagtataagagtaatagtggtggcgataagagagctgtagtagctttagccgacactgaaactggagaagtgtttaagacttcgtttatccgtcaaatagaagtagatgaagaacaattcactaaattgtatctttctaactttgctgcattctttgacctatcacaagcagctattcgggtttttggttactttatgacctgcatgaaacccaaaaatgatttaatcattttcaatagaaaaaaatgcctagaatataccaaatacaaaacagacaaagccgtttataaaggacttgcagaacttgtaaaagctgaaatcatagcccgaggaccagccgataatctttggtttattaatcctctgatagtattcaatggtgaccgagtgacatttgctaaaacatacgtttcggaaaaagactttagctgcccaaaagaaagaagaagcagagaaacgacaattatcacttggctttgatgaacagtaacactccattgagtgaagctgccgtttggtcgctcccctttgggcggggggggatagataaagttcctctatgtaaagttataatgggggatgaaggcaaggtcgctaaccttaccgaggacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtggcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatattcgtttaatatcataaataatttattttattttaaaatgcgcgggtgcaaaggtaagaggttttattttaactaccaaatgttttcggaagttttttcgcttttctttttctatcgtttctcagactctcttagcgaaagggaaagaaggtaaagaagaaaaacaaaacgccttttcttttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacaccatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaattaatgcggctgcaatttttttgggcggggccgcccaaaaaaatcctagcaccctgcagcagtactgcttgaccataagaacaaaaaaacttccgataaagtttggaagataaagctaaaagttcttatctttgcagtatgacgggaatgtaccccaggttttagagctagaaatagcaagttaaaataaggctagtccgttatcaacttgaaaaagtggcaccgagtcggtgctttttttgagatctgtccatacccatgggacgtctgatgattaaggatcttgc

SEQ ID NO: 10 - DNA, 10627 bp, Artificial Sequence
  Description of Artificial Sequence Synthetic polynucleotide
ggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatattcgtttaatatcataaataatttattttattttaaaatgcgcgggtgcaaaggtaagaggttttattttaactaccaaatgttttcggaagttttttcgcttttctttttctatcgtttctcagactctcttagcgaaagggaaagaaggtaaagaagaaaaacaaaacgccttttcttttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacaccatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaattaatgcggctgcaatttttttgggcggggccgcccaaaaaaatcctagcaccctgcagcagtactgcttgaccataagaacaaaaaaacttccgataaagtttggaagataaagctaaaagttcttatctttgcagttgatggagaggtgcaagtaggttttagagctagaaatagcaagttaaaataaggctagtccgttatcaacttgaaaaagtggcaccgagtcggtgctttttttgagatctgtcgactctagaggatccccgggtaccgagctcgaattcactggccgtcgttttacaacgtcgtgactgggaaaaccctggcgttacccaacttaatcgtacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccggtaccgaggacgcgtaaacatttacagttgcatgtggcctattgtttttaagattttactgaagtttgcattaataaagaatatactacagctgatatatgcgcaacatattgtgacgcttgtgatttatttcccttgaaatccttaacaaataccgctaaggtataactgatattattaggcagacatatataggcaacatcattgtgagctgcaagaacaccattttcattaacataacctgaacctgtcttatgcgctataacaaccccttctttatcaagaagtggagctgctatcctatctacacctgttttgcattcttttaacgtattcttaatgaaactttgtttctcatcatcgataagaccttcagtaaacaaacgattcatcaacattgcagcaccaagaggagatgtatagttagagtaagccttgttatggtcagccgacatttcctcttccgtataagctatctgaaaacttgaacgaggaatgagtgtggctataaaactatctgtttgagcgacattaaccatatccttaaacataaggttgcttgcattgttgtcactctgagtaagagtataacgcagcaaatctctcactgtcaatgatatgactggccctgaataatctttcagcataggactccaagtctttgggtcaagtttatccctatttatatttactaaggtatcaagtgaaattcctttattgtcaaagtcattacaaagagctaatgcctgatgaaccttaaacacactcatcataggataaacactcttattattgaccttaaccgtatctctgttattaacaataaccgccacaccaatttcgccaggacaagctgagacaatttgagaaatgctatcagtcaaaacatttgttaaaggaggatttgcgctatcttttgtcgctgatttatggaacaatgaaaataccaagatgaaaatgcaaactaaagctatactcaaaactacgatttgtttttttctgtttttttccattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtgg

SEQ ID NO: 11 - DNA, 20 bp, Artificial Sequence
  Description of Artificial Sequence Synthetic oligonucleotide
atacaggaaaccaatcgtag

SEQ ID NO: 12 - DNA, 20 bp, Artificial Sequence
  Description of Artificial Sequence Synthetic oligonucleotide
ggaagaatcgaagttatatg

SEQ ID NO: 13 - DNA, 20 bp, Artificial Sequence
  Description of Artificial Sequence Synthetic oligonucleotide
aatccactggatgccatccg

SEQ ID NO: 14 - DNA, 20 bp, Artificial Sequence
  Description of Artificial Sequence Synthetic oligonucleotide
gcttatgtctatctatccgg

SEQ ID NO: 15 - DNA, 64 bp, Artificial Sequence
  Description of Artificial Sequence Synthetic oligonucleotide
aaagttcttatctttgcagttgatggagaggtgcaagtaggttttagagctagaaatagcaagt

SEQ ID NO: 16 - DNA, 64 bp, Artificial Sequence
  Description of Artificial Sequence Synthetic oligonucleotide
  modified_base (26)..(26): a, c, t, g, unknown or other
  modified_base (30)..(30): a, c, t, g, unknown or other
  modified_base (38)..(38): a, c, t, g, unknown or other
  modified_base (47)..(47): a, c, t, g, unknown or other
  modified_base (59)..(59): a, c, t, g, unknown or other
aaagttcttatctttgcagttgatgnagangcggtacnaagttttanagctagaaatancaagt

SEQ ID NO: 17 - DNA, 69 bp, Artificial Sequence
  Description of Artificial Sequence Synthetic oligonucleotide
aagctaaaagttcttatctttgcagttgatggagaggtgcaagtaggttttagagctagaaatagcaag

SEQ ID NO: 18 - DNA, 76 bp, Artificial Sequence
  Description of Artificial Sequence Synthetic oligonucleotide
tgaaaaccgttgtactgcgaggtacccggggttcgaatccctgtctctccgcaaaaaacgctgaaaatcagcagat

SEQ ID NO: 19 - DNA, 76 bp, Artificial Sequence
  Description of Artificial Sequence Synthetic oligonucleotide
atctgctgattttcagcgttttttgcggagagacagggattcgaaccccgggtacctcgcagtacaacggttttca

SEQ ID NO: 20 - DNA, 74 bp, Artificial Sequence
  Description of Artificial Sequence Synthetic oligonucleotide
tgaaaaccgttgtactgcgaggtacccggggttcgaatccctgtctctccgcaaaaaacgctgaaaatcagcag

SEQ ID NO: 21 - DNA, 74 bp, Artificial Sequence
  Description of Artificial Sequence Synthetic oligonucleotide
ctgctgattttcagcgttttttgcggagagacagggattcgaaccccgggtacctcgcagtacaacggttttca

SEQ ID NO: 22 - DNA, 72 bp, Artificial Sequence
  Description of Artificial Sequence Synthetic oligonucleotide
aaccgttgtactgcgaggtacccggggttcgaatccctgtctctccgcaaaaaacgctgaaaatcagcagat

SEQ ID NO: 23 - DNA, 72 bp, Artificial Sequence
  Description of Artificial Sequence Synthetic oligonucleotide
atctgctgattttcagcgttttttgcggagagacagggattcgaaccccgggtacctcgcagtacaacggtt

SEQ ID NO: 24 - DNA, 68 bp, Artificial Sequence
  Description of Artificial Sequence Synthetic oligonucleotide
gttgtactgcgaggtacccggggttcgaatccctgtctctccgcaaaaaacgctgaaaatcagcagat

SEQ ID NO: 25 - DNA, 63 bp, Artificial Sequence
  Description of Artificial Sequence Synthetic oligonucleotide
tctgtgaaagtataagttcgagagcctgtctctccgctgaacgtgctggacagaaatggtcag

SEQ ID NO: 26 - DNA, 63 bp, Artificial Sequence
  Description of Artificial Sequence Synthetic oligonucleotide
ctgaccatttctgtccagcacgttcagcggagagacaggctctcgaacttatactttcacaga

SEQ ID NO: 27 - DNA, 46 bp, Artificial Sequence
  Description of Artificial Sequence Synthetic oligonucleotide
tctgtgaaagtataagttcgagagcctgtctctcagaaatggtcag

SEQ ID NO: 28 - DNA, 46 bp, Artificial Sequence
  Description of Artificial Sequence Synthetic oligonucleotide
ctgaccatttctgagagacaggctctcgaacttatactttcacaga

SEQ ID NO: 29 - DNA, 50 bp, Artificial Sequence
  Description of Artificial Sequence Synthetic oligonucleotide
tctgtgaaagtataagctctccgctgaacgtgctggacagaaatggtcag

SEQ ID NO: 30 - DNA, 50 bp, Artificial Sequence
  Description of Artificial Sequence Synthetic oligonucleotide
ctgaccatttctgtccagcacgttcagcggagagcttatactttcacaga

SEQ ID NO: 31 - DNA, 53 bp, Artificial Sequence
  Description of Artificial Sequence Synthetic oligonucleotide
tctgtgaaagtataagttcgagtccgctgaacgtgctggacagaaatggtcag

SEQ ID NO: 32 - DNA, 53 bp, Artificial Sequence
  Description of Artificial Sequence Synthetic oligonucleotide
ctgaccatttctgtccagcacgttcagcggactcgaacttatactttcacaga

SEQ ID NO: 33 - DNA, 48 bp, Artificial Sequence
  Description of Artificial Sequence Synthetic oligonucleotide
tctgtgaaagtataagctccgctgaacgtgctggacagaaatggtcag

SEQ ID NO: 34 - DNA, 48 bp, Artificial Sequence
  Description of Artificial Sequence Synthetic oligonucleotide
ctgaccatttctgtccagcacgttcagcggagcttatactttcacaga

SEQ ID NO: 35 - DNA, 52 bp, Artificial Sequence
  Description of Artificial Sequence Synthetic oligonucleotide
tctgtgaaaggagcctgtctctccgctgaacgtgctggacagaaatggtcag

SEQ ID NO: 36 - DNA, 52 bp, Artificial Sequence
  Description of Artificial Sequence Synthetic oligonucleotide
ctgaccatttctgtccagcacgttcagcggagagacaggctcctttcacaga

SEQ ID NO: 37 - DNA, 58 bp, Artificial Sequence
  Description of Artificial Sequence Synthetic oligonucleotide
tctgtgaaagtataagttcgagagcctgtctctccgctgaacgtgctggacagaaatg

SEQ ID NO: 38 - DNA, 58 bp, Artificial Sequence
  Description of Artificial Sequence Synthetic oligonucleotide
catttctgtccagcacgttcagcggagagacaggctctcgaacttatactttcacaga

(19) United States
(12) Patent Application Publication
(10) Pub. No.: US 2021/0095273 A1
(43) Pub. Date: Apr. 1, 2021

(54) MODULATION OF MICROBIOTA COMPOSITIONS USING TARGETED NUCLEASES

(71) Applicants: SIGMA-ALDRICH CO. LLC, St. Louis, MO (US); WASHINGTON UNIVERSITY, St. Louis, MO (US)

(72) Inventors: Erik Eastlund, Fenton, MO (US); Zhigang Zhang, Creve Coeur, MO (US); Gregory D. Davis, Webster Groves, MO (US); Zachary Walter Beller, St. Louis, MO (US); Jeffrey Ivan Gordon, St. Louis, MO (US)

(21) Appl. No.: 17/037,141

(22) Filed: Sep. 29, 2020

Related U.S. Application Data
(60) Provisional application No. 63/052,825, filed on Jul. 16, 2020.
(60) Provisional application No. 62/909,078, filed on Oct. 1, 2019.
(60) Provisional application No. 62/908,130, filed on Sep. 30, 2019.

(51) Int. Cl. C12N 15/11; C12N 9/22; C12N 15/90; A61K 31/7088; A61K 38/46; A61K 31/65; A61P 35/00; A61P 37/02; A61P 31/04; C12N 15/74

(52) U.S. Cl. CPC C12N 15/11; C12N 9/22; C12N 15/902; A61K 31/7088; A61K 38/465; A61K 31/65; C12N 2800/101; A61P 37/02; A61P 31/04; C12N 15/74; C12N 2310/20; C12N 2800/80; C12N 2840/002; A61P 35/00

(57) ABSTRACT

Compositions and methods for remodeling complex populations of microbes are provided herein. RNA-guided nuclease systems are engineered to target sites in chromosomal DNA of a targeted prokaryotic, wherein the level of targeted prokaryote can be modulated in a mixed population of prokaryotes.

### RELATED APPLICATIONS

The present application claims the benefit of priority of U.S. Provisional Application No. 62/908,130, filed Sep. 30, 2019, and of U.S. Provisional Application No. 62/909,078, filed Oct. 1, 2019, and of U.S. Provisional Application No. 63/052,825, filed Jul. 16, 2020, the entire contents of each of which is incorporated herein by reference.

### SEQUENCE LISTING

This application contains a Sequence Listing that has been submitted in ASCII format via EFS-Web and is hereby incorporated by reference in its entirety. The ASCII copy, created on Sep. 29, 2020 is named P19-171-_US-NP_SL.txt, and is 50,797 bytes in size.

### FIELD

The present disclosure relates to compositions and methods for remodeling the composition of microbiota.

### BACKGROUND

Controlling the composition and expressed functions of microbial populations is a critical aspect of medicine, biotechnology, and environmental cycles. While classic antimicrobial strategies provide some control, what remains elusive is a generalized and programmable strategy that can distinguish between even closely related microorganisms and that allows for fine control over the composition of a microbial population. Recent advances indicate that RNA-guided nuclease systems can be designed to target specific DNA sequences in microbial populations. It would be beneficial to employ similar strategies to target and remove specific species from multi-species bacterial populations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B illustrate targeted microbiota modulation using an integrated, inducible CRISPR system. Expression of the CRISPR system (Cas9 endonuclease and guide RNA) leads to chromosomal breaks and ultimately cell death in bacteria. As such, specific Bacteroides strains harboring an integrated CRISPR cassette with a targeting guide RNA can be eliminated from a mixed population in situ upon anhydrotetracycline (aTc) induction.

FIG. 2 presents a schematic of a CRISPR integration vector. The Cas9 protein is expressed from an anhydrotetracycline (aTc)-inducible promoter. The single guide RNA (N20-sgRNA scaffold) is constitutively expressed from P1 promoter, wherein a 20 nucleotide protospacer sequence (N20) specifies the targeted DNA cleavage in the genome when a PAM is present (NGG in the case of Streptococcus pyogenes CRISPR/Cas9).

FIGS. 3A-3C illustrate chromosomal integration of a CRISPR System in the human gut-derived bacterium, Bacteroides thetaiotaomicron (Bt). FIG. 3A diagrams NBU2 integration mechanism. FIG. 3B shows CRISPR integration to Bt via conjugation. FIG. 3C presents colony PCR screening of CRISPR integrants. PCR A: attBT2-1 locus, outside primers; PCR B: attBT2-2 locus, outside primers; PCR C: attBT2-1 locus, left junction; PCR D: attBT2-2 locus, left junction. M1-M4: four Bt colonies with non-targeting, control gRNA; T1-T4: four Bt colonies with tdk-targeting gRNA; S1-S4: four Bt colonies with susC-targeting gRNA.

FIGS. 4A-4B illustrates induced CRISPR killing of individual Bacteroides strains using integrated CRISPR system. FIG. 4A presents results on blood agar plates. For selected CRISPR integrants (M1 and T1), tube cultures in TYG+Gm 200, Em 25 were diluted and spread (24 h tube culture, 10-6 dilution, 100 μl spread) on BHI blood agar plate (Gm 200, Em 25) supplemented with anhydrotetracycline (aTc) at concentrations of 0 and 100 ng/ml, respectively. Cells were incubated anaerobically at 37° C. for 40 h. FIG. 4B shows results in TYG liquid medium. Selected CRISPR integrants (M1, M2, T1, T3, S1, S2) were grown from fresh colonies in TYG medium anaerobically at 37° C. for 6 h to OD_{600 nm} ˜0.6, 1:100 dilution to fresh TYG liquid medium (Gm 200, Em 25) supplemented with aTc at final concentrations of 0, 10 and 100 ng/ml, respectively. Growth was assessed during culture under anaerobic conditions at 37° C. for 24 h.

FIGS. 5A-5C presents targeted, inducible CRISPR killing of specific Bacteroides strains in a mixed population in vitro. Selected CRISPR integrants (M1, T1, S1) were grown from fresh colonies in TYG medium anaerobically at 37° C. for 6 h to an OD_{600 nm} ˜0.6. Equal volumes of cell cultures (1:100 dilutions) were mixed and added to fresh TYG liquid medium (Gm 200, Em 25) supplemented with aTc at final concentrations of 0, 10 and 100 ng/ml, respectively. These cultures were incubated anaerobically at 37° C. for 24 h. FIG. 5A: OD_{600 nm} measurement. FIG. 5B: PCR amplifying the guide RNA region (primers binding to Cas9 and NBU2 coding sequences, amplicon size of 1.5 kb) was performed for cultures treated with aTc at concentrations of 100 ng/ml, 10 ng/ml and 0 ng/ml followed by Sanger DNA sequencing. Cultures treated with aTc have only non-targeting control gRNA. FIG. 5C: a culture of M1+S1_aTc100 was diluted (10⁻⁶) and spread onto a BHI blood agar plate (Gm 200, Em 25) and incubated anaerobically at 37° C. for 40 h to obtain single colonies. Colony PCR amplifying the guide RNA region was performed for 5 selected single colonies and a scraped mixture from the agar plate, followed by Sanger DNA sequencing, showing all clones that grew harbored only non-targeting, control gRNA.

FIG. 6 illustrates CRISPR integration on the chromosome of Bacteroides vulgatus (Bv). Colonies from each conjugation, Bv.M (labeled VM1, VM2, VM3, VM4, VM5, VM6 and VM7), and susC_Bv (labeled V1, V2, V3, V4, V5), were picked for colony PCR screening. 0, By wild-type strain; M, DNA ladder. PCR A (outside primers, 0.5 or 0 kb) were used to screen integration at the attBv.3-1 locus; PCR B (outside primers, 0.5 or 0 kb) were used to screen integration at the attBv.3-2 locus, and PCR C (outside primers, 0.6 or 0 kb) were used to screen integration at the attBv.3-3 locus. PCR D (an outside primer and an internal primer binding to ermG coding sequence, 0.6 or 0 kb: left junction of attBv.3-1 locus integration) were used to confirm junctions of integrated chromosome and integration plasmid sequences for selected clones. Left panel: Integrated strains with non-targeting, control guide RNA (M); right panel: integrated strains with targeting susC_Bv guide RNA.

FIGS. 7A-7C illustrate the characterization of the growth of B. thetaiotaomicron CRISPR-mutants. FIG. 7A: Plasmid design for engineering a B. thetaiotaomicron VPI-5482 CRISPR mutant. FIG. 7B: Bt mutants, containing either scrambled gRNA or a tdk targeting gRNA, cultured on blood agar plates±200 ng/mL aTc. FIG. 7C: Box plot of time required to achieve OD600=0.2 for Bt CRISPR mutants when grown in LYBHI medium containing 9 ng/mL aTc.

FIGS. 8A-8D illustrate B. thetaiotaomicron knockdown. FIG. 8A: Experimental design. The arrow designates the time of gavage of the consortium into adult male germ-free C57BI/6J mice; each recipient mouse received 0.5% ethanol vehicle on days 1-8 when aTc was not administered. FIGS. 8B,8C: Bt or B. cellulosilyticus relative abundance across time for each treatment condition and aTc exposure shown by horizontal bars. FIG. 8D: Heatmap displaying difference of median relative abundance (%) of each consortium member (column) at each time point (row) in the four-day treatment arm relative to the vehicle control arm.

FIGS. 9A-9B illustrate B. thetaiotaomicron omission. FIG. 9A: Experimental design. The arrow designates the time of introduction of the 13- or 12-member consortia. FIG. 9B: Heatmap displaying difference of median relative abundance (%) for each consortium member (column) at each time point (row) in the 12-member community treatment arm relative to the 13-member (12 strains+Bt) community arm.

FIG. 10 illustrates targeted microbiota modulation using a stably maintained, inducible CRISPR system. Expression of the CRISPR system (Cas9 endonuclease and guide RNA) leads to chromosomal breaks and ultimately cell death in bacteria. As such, specific Bacteroides strains harboring a stably maintained CRISPR cassette with a targeting guide RNA can be eliminated from a mixed population in situ upon anhydrotetracycline (aTc) induction.

FIGS. 11A-11D are photos of blood agar plates. FIG. 11A illustrates the 10⁻⁴ dilution of pRepA-CRISPR targeting susC in Bacteroides thetaiotaomicron with and without aTc induction on blood BHI plates (no aTc on the left and 100 ng/ml aTc on the right). FIG. 11B illustrates the 10⁻⁶ dilution of pRepA-CRISPR targeting susC in Bacteroides thetaiotaomicron with and without aTc induction on blood BHI plates (no aTC on the left and 100 ng/ml aTc on the right). FIG. 11C illustrates the 10⁻⁴ dilution of pRepA-CRISPR non-targeting in Bacteroides thetaiotaomicron with and without aTc induction on blood BHI plates (no aTC on the left and 100 ng/ml aTc on the right). FIG. 11D illustrates the 10⁻⁶ dilution of pRepA-CRISPR non-targeting in Bacteroides thetaiotaomicron with and without aTc induction on blood BHI plates (no aTC on the left and 100 ng/ml aTc on the right).

FIG. 12 illustrates targeted microbiota modulation using an integrated, inducible CRISPR system. Expression of the CRISPR system (Cas9 endonuclease and guide RNA) leads to chromosomal breaks and ultimately cell death in bacteria. As such, specific Bacteroides strains harboring an integrated CRISPR cassette with a targeting guide RNA can be eliminated from a mixed population in situ upon anhydrotetracycline (aTc) induction.

FIGS. 13A-13B illustrates that Plasmid pNBU2-CRISPR.susC_BWH2-19 integrates only in the attBWH2 site in the t-RNA-Ser gene, BcellWH2_RS22795. The 5′ end of the plasmid integration site is shown in FIG. 13A and the 3′ end of the plasmid integration site is shown in FIG. 13B.

FIG. 14 illustrates OD_{600 nm} readings taken after 24 hours of growth as described in Example 13.

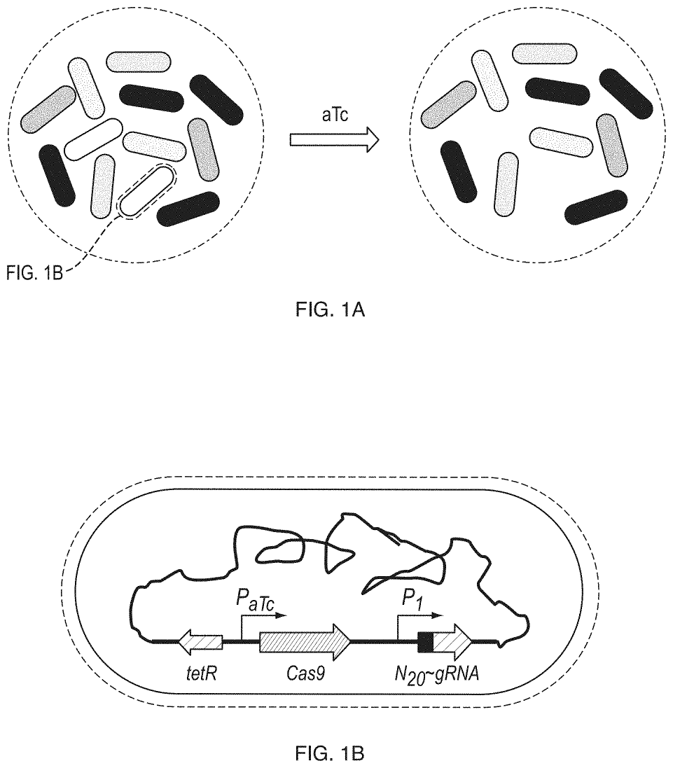

### DETAILED DESCRIPTION

The present disclosure provides engineered RNA-guided nuclease systems that can be used to remodel complex microbial populations by selective knockdown of the abundance of targeted strains. In particular, the RNA-guided nuclease systems are engineered to target sites in chromosomal DNA of the targeted prokaryotic species, where the term “prokaryotic” refers to members of the domains Bacteria and Archaea. The compositions and methods disclosed herein can be used to manipulate microbial community composition ex vivo and within living animals.

### (I) Protein-Nucleic Acid Complexes

One aspect of the present disclosure provides a protein-nucleic acid complex comprising an engineered RNA-guided nuclease system in association with a chromosome of a prokaryote, wherein the engineered RNA-guided nuclease system is targeted to a site in the chromosome, the bacterial chromosome encodes an HU family DNA-binding protein comprising an amino acid sequence having at least 50% sequence identity to the amino acid sequence of SEQ ID NO: 1 (MNKADLISAVAAEAGLSKVDAKKAVEAFVSTVTKALQEGDKVSLIGFGTFSV AERSARTGINPSTKATITIPAKKVTKFKPGAELADAIK), and the chromosome of the bacterial species is associated with HU family DNA-binding proteins have at least 50% sequence identity to the amino acid sequence of SEQ ID NO: 1. In general, the RNA-guided nuclease system that is targeted to the chromosome DNA of the bacterial species is other than a naturally occurring RNA-guided nuclease (e.g., CRISPR) system that is endogenous to the organism of interest.

The RNA-guided nuclease system comprises a DNA endonuclease (e.g., CRISPR nuclease) whose cleavage activity is directed by RNA (e.g., guide RNA). The prokaryote expresses the HU family protein, which associates with the chromosomal DNA of the prokaryote. Thus, the protein-nucleic acid complexes disclosed herein comprise ribonucleoprotein complexes (CRISPR nuclease/gRNA) bound to DNA/protein complexes (prokaryotic chromosomal DNA and associated HU family proteins).

(a) RNA-Guided Nuclease Systems

The protein-nucleic acid complexes disclosed herein comprise an RNA-guided nuclease system, which comprises a DNA endonuclease whose cleavage activity is directed by a guide RNA (gRNA). As detailed below, the gRNA can be engineered to recognize and target a specific sequence in the nucleic acid of interest (e.g., a prokaryotic chromosome).

In general, the RNA-guided endonuclease is a clustered regularly interspaced short palindromic repeats (CRISPR) nuclease. The CRISPR nuclease can be bacterial or archaeal. In some situations, the CRISPR nuclease can be from a Type I CRISPR system, a type II CRISPR system, a type III CRISPR system, a Type IV CRISPR system, a type V CRISPR system, or a type VI CRISPR system. In specific embodiments, the CRISPR nuclease can be from single-subunit effector systems such as Type II, Type V, or Type VI systems. In various embodiments, the CRISPR nuclease can be a Type II Cas9 nuclease, a Type V Cas12 (formerly called Cpf1) nuclease, a Type VI Cas13 (formerly called C2cd) nuclease, a CasX nuclease, or a CasY nuclease.

The CRISPR nuclease can be from Acaryochloris spp., Acetohalobium spp., Acidaminococcus spp., Acidithiobacillus spp., Acidothermus spp., Akkermansia spp., Alicyclobacillus spp., Allochromatium spp., Ammonifex spp., Anabaena spp., Arthrospira spp., Bacillus spp., Bifidobacterium spp., Burkholderiales spp., Caldicelulosiruptor spp., Campylobacter spp., Candidatus spp., Clostridium spp., Corynebacterium spp., Crocosphaera spp., Cyanothece spp., Deltaproteobacterium spp., Exiguobacterium spp., Finegoldia spp., Francisella spp., Ktedonobacter spp., Lachnospiraceae spp., Lactobacillus spp., Leptotrichia spp., Lyngbya spp., Marinobacter spp., Methanohalobium spp., Microscilla spp., Microcoleus spp., Microcystis spp., Mycoplasma spp., Natranaerobius spp., Neisseria spp., Nitratifractor spp., Nitrosococcus spp., Nocardiopsis spp., Nodularia spp., Nostoc spp., Oenococcus spp., Oscillatoria spp., Parasutterella spp., Pelotomaculum spp., Petrotoga spp., Planctomyces spp., Polaromonas spp., Prevotella spp., Pseudoalteromonas spp., Ralstonia spp., Ruminococcus spp., Staphylococcus spp., Streptococcus spp., Streptomyces spp., Streptosporangium spp., Synechococcus spp., Thermosipho spp., Verrucomicrobia spp., or Wolinella spp.

In some aspects, the CRISPR nuclease can be Streptococcus pyogenes Cas9, Francisella novicida Cas9, Staphylococcus aureus Cas9, Streptococcus thermophilus Cas9, Streptococcus pasteurianus Cas9, Campylobacter jejuni Cas9, Neisseria meningitis Cas9, Neisseria cinerea Cas9, Francisella novicida Cas12, Acidaminococcus sp. Cas12, Lachnospiraceae bacterium ND2006 Cas12a, Leptotrichia wadeii Cas13a, Leptotrichia shahii Cas13a, Prevotella sp. P5-125 Cas13, Ruminococcus flavefaciens Cas13d, Deltaproteobacterium CasX, Planctomyces CasX, or Candidatus CasY.

The CRISPR nuclease can be a wild type or naturally-occurring protein. Wild-type CRISPR nucleases generally comprise two nuclease domains, e.g., Cas9 nucleases comprise RuvC and HNH domains, each of which cleaves one strand of a double-stranded sequence. CRISPR nucleases also comprise domains that interact with the guide RNA (e.g., REC1, REC2) or the RNA/DNA heteroduplex (e.g., REC3), and a domain that interacts with the protospacer-adjacent motif (PAM) (i.e., PAM-interacting domain).

Alternatively, the CRISPR nuclease can be modified to have improved targeting specificity, improved fidelity, altered PAM specificity, decreased off-target effects, and/or increased stability. For example, the CRISPR nuclease can be modified to comprise one or more mutations (i.e., substitution, deletion, and/or insertion of at least one amino acid). Non-limiting examples of one or more mutations that improve targeting specificity, improve fidelity, and/or decrease off-target effects include N497A, R661A, Q695A, K810A, K848A, K855A, Q926A, K1003A, R1060A, and/or D1135E (with reference to the numbering system of SpyCas9).

In various embodiments, the CRISPR nuclease can be a nuclease (i.e., cleave both strands of a double-stranded nucleotide sequence or cleave a single-stranded nucleotide sequence). In other embodiment, CRISPR nuclease can be a nickase, which cleaves one strand of a double-stranded sequence. The nickase can be engineered via inactivation of one of the nuclease domains of the CRISPR nuclease. For example, the RuvC domain of a Cas9 protein can be inactivated by mutations such as D10A, D8A, E762A, and/or D986A, or the HNH of a Cas9 protein domain can be inactivated by mutations such as H840A, H559A, N854A, N856A, and/or N863A (with reference to the numbering system of Streptococcus pyogenes Cas9, SpyCas9) to generate a Cas9 nickase (e.g., nCas9). Comparable mutations in other CRISPR nucleases can generate nickases (e.g., nCas12).

A CRISPR system also comprises a guide RNA. A guide RNA interacts with the CRISPR nuclease and a target sequence in the nucleic acid of interest and guides the CRISPR nuclease to the target sequence. The target sequence has no sequence limitation except that the sequence is adjacent to a protospacer adjacent motif (PAM) sequence. Different CRISPR nucleases recognize different PAM sequences. For example, PAM sequences for Cas9 proteins include 5′-NGG, 5′-NGGNG, 5′-NNAGAAW, 5′-NNNNGATT, and 5-NNNNRYAC, and PAM sequences for Cas12 proteins include 5′-TTN and 5′-TTTV, wherein N is defined as any nucleotide, R is defined as either G or A, W is defined as either A or T, Y is defined an either C or T, and V is defined as A, C, or G. In general, Cas9 PAMs are located 3′ of the target sequence, and Cas12 PAMs are located 5′ of the target sequence.

Guide RNAs are engineered to complex with specific CRISPR nucleases. In general, a guide RNA comprises (i) a CRISPR RNA (crRNA) that contains a guide or spacer sequence at the 5′ end that hybridizes at the target site, and (ii) a transacting crRNA (tracrRNA) sequence that interacts with the CRISPR nuclease. The guide or spacer sequence of each guide RNA is different (i.e., is sequence specific). The rest of the guide RNA sequence is generally the same in guide RNAs designed to complex with a specific CRISPR nuclease.

The crRNA comprises the guide sequence at the 5′ end, as well as additional sequence at the 3′ end that base-pairs with sequence at the 5′ end of the tracrRNA to form a duplex structure, and the tracrRNA comprises additional sequence that forms at least one stem-loop structure, which interacts with the CRISP nuclease. The guide RNA can be a single molecule (e.g., a single guide RNA (sgRNA) or 1-piece sgRNA), wherein the crRNA sequence is linked to the tracrRNA sequence. Alternatively, the guide RNA can be two separate molecules (e.g., 2-piece gRNA) comprising a crRNA and a tracrRNA.

The crRNA guide sequence is designed to hybridize with the complement of a target sequence (i.e., protospacer) in the nucleic acid of interest. In general, the complementarity between the guide sequence and the target sequence is at least 80%, at least 85%, at least 90%, at least 95%, or at least 99%. In specific embodiments, the complementarity is complete (i.e., 100%). In various embodiments, the length of the crRNA guide sequence can range from about 15 nucleotides to about 25 nucleotides. For example, the crRNA guide sequence can be about 15, 16, 17, 18, 19, 20, 21, 22, 23, 24, or 25 nucleotides in length. In specific embodiments, the guide is about 19, 20, or 21 nucleotides in length. In one embodiment, the crRNA guide sequence has a length of 20 nucleotides. In certain embodiments, the crRNA can comprise additional 3′ sequence that interacts with tracrRNA. The additional sequence can comprise from about 10 to about 40 nucleotides. In embodiments in which the guide RNA comprises a single molecule, the crRNA and tracrRNA portions of the gRNA can be linked by sequence that forms a loop. The sequence that form the loop can range in length from about 4 nucleotides to about 10 or more nucleotides.

As mentioned above, the tracrRNA comprises repeat sequences that form at least one stem loop structure, which interacts with the CRISPR nuclease. The length of each loop and stem can vary. For example, the loop can range from about 3 to about 10 nucleotides in length, and the stem can range from about 6 to about 20 base pairs in length. The stem can comprise one or more bulges of 1 to about 10 nucleotides. The tracrRNA sequence in the guide RNA generally is based upon the sequence of wild type tracrRNA that interact with the wild-type CRISPR nuclease. The wild-type sequence can be modified to facilitate secondary structure formation, increased secondary structure stability, and the like. For example, one or more nucleotide changes can be introduced into the guide RNA sequence. The tracrRNA sequence can range in length from about 50 nucleotides to about 300 nucleotides. In various embodiments, the tracrRNA can range in length from about 50 to about 90 nucleotides, from about 90 to about 110 nucleotides, from about 110 to about 130 nucleotides, from about 130 to about 150 nucleotides, from about 150 to about 170 nucleotides, from about 170 to about 200 nucleotides, from about 200 to about 250 nucleotides, or from about 250 to about 300 nucleotides. The tracrRNA can comprise an optional extension at the 3′ end of the tracrRNA.

The guide RNA can comprise standard ribonucleotides and/or modified ribonucleotides. In some embodiment, the guide RNA can comprise standard or modified deoxyribonucleotides. In embodiments in which the guide RNA is enzymatically synthesized (i.e., in vivo or in vitro), the guide RNA generally comprises standard ribonucleotides. In embodiments in which the guide RNA is chemically synthesized, the guide RNA can comprise standard or modified ribonucleotides and/or deoxyribonucleotides. Modified ribonucleotides and/or deoxyribonucleotides include base modifications (e.g., pseudouridine, 2-thiouridine, N6-methyladenosine, and the like) and/or sugar modifications (e.g., 2′-O-methy, 2′-fluoro, 2′-amino, locked nucleic acid (LNA), and so forth). The backbone of the guide RNA can also be modified to comprise phosphorothioate linkages, boranophosphate linkages, or peptide nucleic acids.

The guide RNA of a CRISPR nuclease system is engineered to target the CRISPR nuclease system to a specific site in prokaryotic chromosomal DNA such that the protein-nucleic acid complexes, as described above, can be formed. In general, the protein-nucleic acid complex is formed within the prokaryote.

In some embodiments, the engineered CRISPR nuclease system can be integrated into and expressed from the chromosome of the prokaryote. In other embodiments, the engineered CRISPR nuclease system can be carried on and expressed from an extrachromosomal vector. Expression of the engineered CRISPR nuclease system can be regulated. For example, the expression of the engineered CRISPR nuclease system can be regulated by an inducible promoter.

(b) Prokaryotic Chromosome

The protein-nucleic acid complex disclosed herein further comprises a prokaryotic chromosome, wherein the prokaryotic chromosome encodes HU family DNA-binding protein comprising an amino acid sequence with at least 50% sequence identity to the amino acid sequence of SEQ ID NO:1, and the chromosomal DNA of the prokaryote is associated with said HU family DNA-binding protein. The HU family of DNA-binding proteins comprises small (˜90 amino acids) basic histone-like proteins that bind double stranded DNA without sequence specificity and bind DNA structures such as forks, three/four way junctions, nicks, overhangs, and bulges. Binding of HU family DNA-binding proteins can stabilize the DNA and protect it from denaturation under extreme environmental conditions.

The chromosome can be within members of the domain Bacteria or the domain Archaea. In some embodiments, the organism is a bacterial species or different strains of that species. In some embodiments, the HU family DNA-binding protein comprises an amino acid sequence having at least 55%, at least 60%, at least 65%, at least 70%, at least 75%, at least 80%, at least 85%, at least 90%, at least 95%, or at least 99% sequence identity to SEQ ID NO:1.

In specific embodiments, the prokaryote is a member of the genus Bacteroides. Bacteroides species are prominent anaerobic symbionts of mammalian gut microbiota. They contain a variety of saccharolytic enzymes and are the primary fermenters of polysaccharides in the gut. They maintain complex and generally beneficial relationships with the host when retained in the gut, but can cause significant pathology if they escape this environment. Non-limiting examples of Bacteroides species include B. acidifaciens, B. bacterium, B. barnesiaes, B. caccae, B. caecicola, B. caecigallinarum, B. capillosis, B. cellulosilyticus, B. cellulosolvens, B. clarus, B. coagulans, B. coprocola, B. coprophilus, B. coprosuis, B. distasonis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. heparinolyticus, B. intestinalis, B. johnsonii, B. luti, B. massiliensis, B. melaninogenicus, B. neonati, B. nordii, B. oleiciplenus, B. oris, B. ovatus, B. paurosaccharolyticus, B. plebeius, B. polypragmatus, B. pro pionicifaciens, B. putredinis, B. pyogenes, B. reticulotermitis, B. rodentium, B. salanitronis, B. salyersiae, B. sartorii, B. sediment, B. stercoris, B. stercorirosoris, B. suis, B. tectus, B. thetaiotaomicron, B. timonensis, B. uniformis, B. vulgatus, B. xylanisolvens, B. xylanolyticus., and B. zoogleoformans.

In some embodiments, the prokaryotic chromosome is a chromosome chosen from Bacteroides thetaiotaomicron, Bacteroides vulgatus, Bacteroides cellulosilyticus, Bacteroides fragilis, Bacteroides helcogenes, Bacteroides ovatus, Bacteroides salanitronis, Bacteroides uniformis, or Bacteroides xylanisolvens.

In some embodiments, the chromosome is chosen from Barnesiella sp., Barnesiella viscericola, Capnocytphaga sp., Odoribacter splanchnicus, Paludibacter sp., Parabacteroides sp., Porphyromonadaceae bacterium, and Schleiferia sp.

(c) Specific Protein-Nucleic Acid Complexes

In specific embodiments, the protein-nucleic acid complex can comprise an engineered CRISPR Cas9/gRNA system or an engineered CRISPR Cas12/gRNA system bound to or associated with a Bacteroides chromosome.

### (II) Methods for Generating the Protein-Nucleic Acid Complexes

A further aspect of the present disclosure provides methods for generating complexes comprising an engineered RNA-guided (CRISPR) nuclease system and a prokaryotic chromosome encoding an HU family DNA-binding protein as described above. Said methods comprise (a) engineering the CRISPR nuclease system to target a site in the prokaryote chromosome, and (b) introducing the engineered CRISPR nuclease system into the prokaryote.

Engineering the CRISPR nuclease system comprises designing a guide RNA whose crRNA guide sequence targets a specific (˜19-22 nt) sequence in the prokaryotic chromosome that is adjacent to a PAM sequence (which is recognized by the CRISPR nuclease of interest) and whose tracrRNA sequence is recognized by the CRISPR nuclease of interest, as described above in section (I)(a). The engineered CRISPR system can be introduced into the prokaryote as an encoding nucleic acid. For example, the encoding nucleic acid can be part of a vector. Means for delivering or introducing various vectors into are well known in the art.

The vector encoding the engineered CRISPR system (i.e., CRISPR nuclease and guide RNA) can be a plasmid vector, phagemid vector, viral vector, bacteriophage vector, bacteriophage-plasmid hybrid vector, or other suitable vector. The vector can be an integrative vector, a conjugation vector, a shuttle vector, an expression vector, an extrachromosomal vector, and so forth.

The nucleic acid sequence encoding the CRISPR nuclease can be operably linked to a promoter for expression in the prokaryote. In specific embodiments, the promoter operably linked to the engineered CRISPR nuclease can be a regulated promoter. In some aspects, the regulated promoter can be regulated by a promoter inducing chemical. In such embodiments, the promoter can be pTetO, which is based on the Escherichia coli Tn10-derived tet regulatory system and consists of a strong tet operator (tetO)-containing mycobacterial promoter and expression cassette for the repressor (TetR) and the promoter inducing chemical can be anhydrotetracycline (aTc). In other embodiments, the promoter can be pBAD or araC-ParaBAD and the promoter inducing chemical can be arabinose. In further embodiments, the promoter can be pLac or tac (trp-lac) and the promoter inducing chemical can be lactose/IPTG. In other embodiments, the promoter can be pPrpB and the promoter inducing chemical can be propionate.

The nucleic acid sequence encoding the at least one guide RNA can be operably linked to a promoter for expression in the prokaryote of interest. In embodiments in which the prokaryote of interest is Bacteroides, the constitutive promoter can be the P1 promoter, which lies upstream of the B. thetaiotaomicron 16S rRNA gene BT_r09 (Wegmann et al., Applied Environ. Microbiol., 2013, 79:1980-1989). Other suitable Bacteroides promoters include P2, P1T_{D}, P1T_{P}, P1T_{DP} (Lim et al., Cell, 2017, 169:547-558), P_{AM}, P_{cfiA}, P_{cepA}, P_{BT}1311 (Mimee et al., Cell Systems, 2015, 1:62-71) or variants of any of the foregoing promoters. In other embodiments, the constitutive promoter can be an E. coli σ⁷⁰ promoter or derivative thereof, a B. subtilis σ^{A} promoter or derivative thereof, or a Salmonella Pspv2 promoter or derivative thereof. Persons skilled in the art are familiar with additional constitutive promoters that are suitable for the prokaryote of interest.

In some embodiments, the vector can be an integrative vector and can further comprise sequence encoding a recombinase, as well as one or more recombinase recognition sites. In general, the recombinase is an irreversible recombinase. Non-limiting examples of suitable recombinases include the Bacteroides intN2 tyrosine integrase (coded by NBU2 gene), Streptomyces phage phiC31 (φC31) recombinase, coliphage P4 recombinase, coliphage lambda integrase, Listeria A118 phage recombinase, and actinophage R4 Sre recombinase. Recombinases/integrases mediate recombination between two sequence specific recognition (or attachment) sites (e.g., an attP site and an attB site). In some embodiments, the vector can comprise one of the recombinase recognition sites (e.g., attP) and the other recombinase recognition site (e.g., attB) can be located in the chromosome of the prokaryote (e.g., near a tRNA-ser gene). In such situations, the entire vector can be integrated into the chromosome of the prokaryote. In other embodiments, the sequence encoding the engineered CRISPR nuclease system can be flanked by the two recombinase recognition sites, such that only the sequence encoding the engineered CRISPR nuclease system is integrated into the prokaryotic chromosome.

Any of the vectors described above can further comprise at least one transcriptional termination sequence, as well as at least one origin of replication and/or at least one selectable marker sequence (e.g., antibiotic resistance genes) for propagation and selection in prokaryotic cells of interest.

Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In embodiments in which the vector encoding the engineered CRISPR nuclease system is an integrative vector, the nucleic acid encoding the engineered CRISPR system (or the entire vector) can be stably integrated into the bacterial chromosome after delivery of the vector to the bacterium (and expression of the recombinase/integrase). In embodiments in which the vector encoding the engineered CRISPR nuclease system is not an integrative vector, the vector can remain extrachromosomal after delivery of the vector to the microbe.

In embodiments in which the sequence encoding the CRISPR nuclease is operably linked to an inducible promoter, expression of the CRISPR nuclease system can be regulated by introducing the promoter inducing chemical into the prokaryote. In specific embodiments, the promoter inducing chemical can be anhydrotetracycline. Upon induction, the CRISPR nuclease is synthesized and complexes with the at least one guide RNA, which targets the CRISPR nuclease system to the target site in the bacterial chromosome, thereby forming the protein-nucleic acid complex as disclosed herein.

### (III) Methods for Modulating Microbiota Compositions

A further aspect of the present disclosure encompasses methods for altering the population and composition of microbiota, by selectively slowing the growth of a target microbe (prokaryote) in a mixed population of microbes. The method comprises expressing an engineered RNA-guided (CRISPR) nuclease system in the target prokaryote, wherein the engineered RNA-guided nuclease system is targeted to a site in a chromosome of the target prokaryote such that at least one double strand break is introduced in the chromosome of the target prokaryote, thereby slowing the growth or propagation of the target prokaryotes. The growth of the target prokaryote comprising at least one double strand break in chromosomal DNA is slowed or halted because DNA breaks generally are not repaired or are inefficiently repaired in prokaryotes. Slowing the growth of the target prokaryote leads to reduced or eliminated levels of the target prokaryote in the mixed population of prokaryotes.

Any of the CRISPR nuclease systems described above in section (I)(a) can be engineered as described above in section (II) to target a site in the chromosome of a prokaryote of interest, which are described above in section (I)(b). The engineered CRISPR nuclease system can be introduced as part of a vector into the prokaryote as described above in section (II). In general, the CRISPR nuclease is inducible (i.e., its encoding sequence is operably linked to an inducible promoter). As such, the CRISPR nuclease can be expressed at a defined point in time. In the absence of a promoter inducing chemical, the CRISPR nuclease system cannot be generated. A CRISPR nuclease can be produced by exposing the prokaryote to a promoter inducing chemical, such that the CRISPR nuclease is expressed from the chromosomally integrated encoding sequence or the extrachromosomal encoding sequence as described above in section (II). The CRISPR nuclease complexes with the at least one guide RNA that is constitutively expressed from the chromosomally integrated encoding sequence or the extrachromosomal encoding sequence, thereby forming an active CRISPR nuclease system. The CRISPR nuclease system is targeted to the target site in the prokaryotic chromosome, where it introduces a double strand break in the chromosomal DNA. The double strand break results in slowed growth and/or death of the target prokaryote. As a consequence, the mixed population of prokaryotes has reduced or eliminated levels of the target prokaryote.

In some embodiments, the target prokaryote can be a Bacteroides species, as detailed above in section (I)(b).

The engineered CRISPR system can be introduced into the target prokaryote within the mixed population of prokaryotes. Alternatively, the engineered CRISPR system can be introduced into the target prokaryote, which is then mixed with the mixed population of prokaryotes.

In some embodiments, the mixed population of prokaryotes can be harbored in cell culture, wherein exposure to the promoter inducing chemical leads to reduced or eliminated levels of the target prokaryote.

In other embodiments, the mixed population of prokaryotes can be harbored in a mammal's digestive tract (or gut), wherein administration of the promoter inducing chemical leads to reduced or eliminated levels of the target prokaryote in the gut microbiota. The promoter inducing chemical can be administered orally (e.g., via food, drink, or a pharmaceutical formulation). The mammal can be a mouse, rat, or other research animal. In specific embodiments, the mammal can be a human. Reduction or elimination of the target prokaryote (e.g., Bacteroides) can lead to improved gut health.

The mixed population of prokaryotes (in cell culture or a digestive tract) can comprise a wide diversity of taxa. For example, human gut microbiota can comprise hundreds of different species of bacteria and many strain-level variants of these species.

In certain embodiments, the mammal (e.g., human) can be undergoing cancer immunotherapy, wherein immunotherapy responders have been shown to have lower levels of Bacteroides species in their gut microbiota as compared to non-responders (Gopalakrishnan et al., Science, 2018, 359:97-103). Thus, reduction in the levels of Bacteroides species in gut microbiota may lead to better human cancer immunotherapy outcomes.

In certain embodiments, the mammal (e.g., human, canine, feline, porcine, equine, or bovine) can undergo gut surgery for a variety of reasons including, but not limited to, inflammatory bowel disease, Crohn's disease, diverticulitis, bowel blockage, polyp removal, cancerous tissue removal, ulcerative colitis, bowel resection, proctectomy, complete colectomy, or partial colectomy wherein attenuation of Bacteroides fragilis species within the mammalian gut pre-surgery by an inducible CRISPR system may reduce the risk of post-surgery infections by B. fragilis at locations outside the gut, but within the mammalian body. Locations outside the gut include the external surface of the gut. The inducible CRISPR systems within B. fragilis can be targeted to cut or modify a location similar, but not limited to, a pathogenicity island, toxins (i.e., B. fragilis toxin or BFT) or other unique sequence associated with infectious strains of B. fragilis or other native gut prokaryotes known to cause post-surgical infections. For example, levels of nontoxigenic B. fragilis (NTBF) and enterotoxigenic B. fragilis (ETBF) maybe be selectively modulated using engineered inducible CRISPR systems placed within ETBF strains, but not NTBF strains. Other bacterial taxa that cause infections after gut surgery may include Bacteroides capillosis, Escherchia coli, Enterococcus faecalis, Gamella haemolysan, and Morganella morganii. Delivery of the inducible CRISPR system to the gut microbiota may occur as part of a probiotic treatment before, during, or after surgery. Delivery of the inducible CRISPR system to the target prokaryote may occur outside the mammalian body or within the mammalian body. Delivery of the inducble CRISPR system to the target prokaryote may occur via nucleic acid vectors such as plasm ids or bacteriophage. Delivery of plasm ids may occur via electroporation, chemical transformation, or bacteria-to-bacteria conjugation.

In various embodiments, the level of the target prokaryote can be reduced by at least about 30%, at least about 40%, at least about 50%, at least about 60%, at least about 70%, at least about 80%, at least about 90%, or at least about 99% relative to that before expression of the CRISPR nuclease. In certain embodiments, the target prokaryote can be reduced to undetectable levels in the mixed population of prokaryotes after expression of the CRISPR nuclease.

### (IV) CRISPR Integrated Prokaryotes as Probiotics

Yet another aspect of the present disclosure encompasses engineered prokaryotes for use as probiotics. The engineered prokaryotes comprise any of engineered CRISPR nuclease systems described in section (I) integrated into the prokaryotic chromosome or maintained as episomal vectors within the prokaryotic cell. In some embodiments, the engineered prokaryote is an engineered Bacteroides comprising an inducible CRISPR nuclease system. Administration of the engineered Bacteroides to a mammalian subject followed by induction of the CRISPR system can be used to reduce the relative abundance of Bacteroides strains in gut microbiota. In other embodiments, the Bacteroides strains can be engineered to out-compete wildtype strains of Bacteroides in gut microbiota. In these and other embodiments, engineered Bacteroides strains providing a therapeutic benefit for the mammalian subject can then be removed from the mammalian subject by induction of the inducible CRISPR nuclease system.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

The term “expression” with respect to a gene or polynucleotide refers to transcription of the gene or polynucleotide and, as appropriate, translation of an mRNA transcript to a protein or polypeptide. Thus, as will be clear from the context, expression of a protein or polypeptide results from transcription and/or translation of the open reading frame.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The term “nuclease,” which is used interchangeably with the term “endonuclease,” refers to an enzyme that cleaves both strands of a double-stranded nucleic acid sequence or cleaves a single-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The terms “target sequence,” and “target site” are used interchangeably to refer to the specific sequence in the nucleic acid of interest (e.g., chromosomal DNA or cellular RNA) to which the CRISPR system is targeted, and the site at which the CRISPR system modifies the nucleic acid or protein(s) associated with the nucleic acid.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1

### Vector Construction

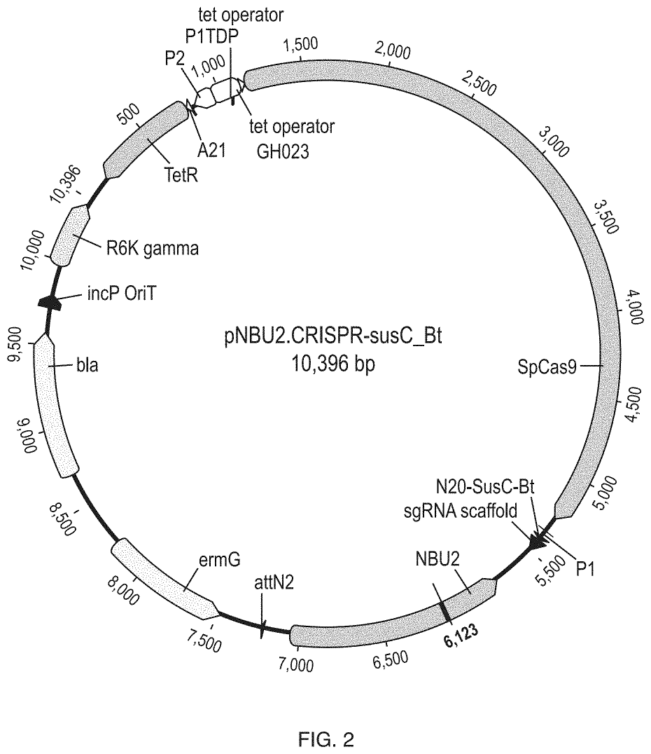

The CRISPR integration pNBU2-CRISPR plasmids were constructed using Gibson cloning (NEBuild HIFI DNA Assembly Master Mix, New England Biolabs) of plasmid backbone (RP4-oriT, R6K ori, bla, ermG) from pExchange-tdk, NBU2 integrase from pNBU2-tetQb, and anhydrotetracycline (aTc) inducible CRISPR cassettes (P2-A21-tetR, P1TDP-GH023-SpCas9, P1-N20 sgRNA scaffold) assembled from synthetic DNAs or PCR of genomic DNA of Streptococcus pyogenes strain SF370. FIG. 2 illustrates the plasmid design.

The plasmid backbone harbors R6K origin of replication and bla sequence for ampicillin selection in E. coli, RP4-oriT sequence for conjugation and ermG sequence for erythromycin (Em) selection in Bacteroides. NBU2 encodes the intN2 tyrosine integrase, which mediates sequence-specific recombination between the attN2 site on pNBU2-CRISPR plasmid and one of the attB sites located on the chromosome of Bacteroides cells. The attN2 and attB have the same 13 bp recognition nucleotide sequence (5′-3′): CCTGTCTCTCCGC (SEQ ID NO: 2).

The inducible CRISPR cassettes include aTc inducible SpCas9 under the control of TetR regulator (P2-A21-tetR, P1TDP-GH023-SpCas9), and constitutively expressed guide RNA under P1 promoter (P1-N20 sgRNA scaffold). The promoters and ribosomal binding sites are derived and engineered from regulatory sequences of Bacteroides thetaiotaomicron 16S rRNA genes, as described in Lim et al., Cell, 2017, 169:547-558. The guide RNA is a nucleotide sequence that is homologous to a coding DNA sequence, or non-coding DNA sequence, or a non-targeting scramble nucleotide sequence. This sequence can be of any form as long as it is compatible with protospacer adjacent motif (PAM) requirements of different Cas9 homologs. The guide RNA can be either in separate transcriptional units of tracrRNA and crRNA or fused into a hybrid chimeric tracr/crRNA single guide(sgRNA).

The DNA sequence for the above plasmid is presented in SEQ ID NO: 3:

Plasmid (pNBU2.CRISPR-susC_Bt)
DNA sequence (10,396 bp)
(SEQ ID NO: 3)
GGAAAGCGGGCAGTGAGCGCAACGCAATTAATGTGAGTTAGCTCACT
CATTAGGCACCCCAGGCTTTACACTTTATGCTTCCGGCTCGTATGTT
GTGTGGAATTGTGAGCGGATAACAATTTCACACAGGAAACAGCTATG
ACCATGATTACGCCCTTAAGACCCACTTTCACATTTAAGTTGTTTTT
CTAATCCGCATATGATCAATTCAAGGCCGAATAAGAAGGCTGGCTCT
GCACCTTGGTGATCAAATAATTCGATAGCTTGTCGTAATAATGGCGG
CATACTATCAGTAGTAGGTGTTTCCCTTTCTTCTTTAGCGACTTGAT
GCTCTTGATCTTCCAATACGCAACCTAAAGTAAAATGCCCCACAGCG
CTGAGTGCATATAATGCATTCTCTAGTGAAAAACCTTGTTGGCATAA
AAAGGCTAATTGATTTTCGAGAGTTTCATACTGTTTTTCTGTAGGCC
GTGTACCTAAATGTACTTTTGCTCCATCGCGATGACTTAGTAAAGCA
CATCTAAAACTTTTAGCGTTATTACGTAAAAAATCTTGCCAGCTTTC
CCCTTCTAAAGGGCAAAAGTGAGTATGGTGCCTATCTAACATCTCAA
TGGCTAAGGCGTCGAGCAAAGCCCGCTTATTTTTTACATGCCAATAC
AATGTAGGCTGCTCTACACCTAGCTTCTGGGCGAGTTTACGGGTTGT
TAAACCTTCGATTCCGACCTCATTAAGCAGCTCTAATGCGCTGTTAA
TCACTTTACTTTTATCTAATCTAGACATATTCGTTTAATATCATAAA
TAATTTATTTTATTTTAAAATGCGCGGGTGCAAAGGTAAGAGGTTTT
ATTTTAACTACCAAATGTTTTCGGAAGTTTTTTCGCTTTTCTTTTTC
TATCGTTTCTCAGACTCTCTTAGCGAAAGGGAAAGAAGGTAAAGAAG
AAAAACAAAACGCCTTTTCTTTTTTGCACCCGCTTTCCAAGAGAAGA
AAGCCTTGTTAAATTGACTTAGTGTAAAAGCGCAGTACTGCTTGACC
ATAAGAACAAAAAAATCTCTATCACTGATAGGGATAAAGTTTGGAAG
ATAAAGCTAAAAGTTCTTATCTTTGCAGTCTCCCTATCAGTGATAGA
GACGAAATAAAGACATATAAAAGAAAAGACACCATGGATAAGAAATA
CTCAATAGGCTTAGATATCGGCACAAATAGCGTCGGATGGGCGGTGA
TCACTGATGAATATAAGGTTCCGTCTAAAAAGTTCAAGGTTCTGGGA
AATACAGACCGCCACAGTATCAAAAAAAATCTTATAGGGGCTCTTTT
ATTTGACAGTGGAGAGACAGCGGAAGCGACTCGTCTCAAACGGACAG
CTCGTAGAAGGTATACACGTCGGAAGAATCGTATTTGTTATCTACAG
GAGATTTTTTCAAATGAGATGGCGAAAGTAGATGATAGTTTCTTTCA
TCGACTTGAAGAGTCTTTTTTGGTGGAAGAAGACAAGAAGCATGAAC
GTCATCCTATTTTTGGAAATATAGTAGATGAAGTTGCTTATCATGAG
AAATATCCAACTATCTATCATCTGCGAAAAAAATTGGTAGATTCTAC
TGATAAAGCGGATTTGCGCTTAATCTATTTGGCCTTAGCGCATATGA
TTAAGTTTCGTGGTCATTTTTTGATTGAGGGAGATTTAAATCCTGAT
AATAGTGATGTGGACAAACTATTTATCCAGTTGGTACAAACCTACAA
TCAATTATTTGAAGAAAACCCTATTAACGCAAGTGGAGTAGATGCTA
AAGCGATTCTTTCTGCACGATTGAGTAAATCAAGACGATTAGAAAAT
CTCATTGCTCAGCTCCCCGGTGAGAAGAAAAATGGCTTATTTGGGAA
TCTCATTGCTTTGTCATTGGGTTTGACCCCTAATTTTAAATCAAATT
TTGATTTGGCAGAAGATGCTAAATTACAGCTTTCAAAAGATACTTAC
GATGATGATTTAGATAATTTATTGGCGCAAATTGGAGATCAATATGC
TGATTTGTTTTTGGCAGCTAAGAATTTATCAGATGCTATTTTACTTT
CAGATATCCTAAGAGTAAATACTGAAATAACTAAGGCTCCCCTATCA
GCTTCAATGATTAAACGCTACGATGAACATCATCAAGACTTGACTCT
TTTAAAAGCTTTAGTTCGACAACAACTTCCAGAAAAGTATAAAGAAA
TCTTTTTTGATCAATCAAAAAACGGATATGCAGGTTATATTGATGGG
GGAGCTAGCCAAGAAGAATTTTATAAATTTATCAAACCAATTTTAGA
AAAAATGGATGGTACTGAGGAATTATTGGTGAAACTAAATCGTGAAG
ATTTGCTGCGCAAGCAACGGACCTTTGACAACGGCTCTATTCCCCAT
CAAATTCACTTGGGTGAGCTGCATGCTATTTTGAGAAGACAAGAAGA
CTTTTATCCATTTTTAAAAGACAATCGTGAGAAGATTGAAAAAATCT
TGACTTTTCGAATTCCTTATTATGTTGGTCCATTGGCGCGTGGCAAT
AGTCGTTTTGCATGGATGACTCGGAAGTCTGAAGAAACAATTACCCC
ATGGAATTTTGAAGAAGTTGTCGATAAAGGTGCTTCAGCTCAATCAT
TTATTGAACGCATGACAAACTTTGATAAAAATCTTCCAAATGAAAAA
GTACTACCAAAACATAGTTTGCTTTATGAGTATTTTACGGTTTATAA
CGAATTGACAAAGGTCAAATATGTTACTGAAGGAATGCGAAAACCAG
CATTTCTTTCAGGTGAACAGAAGAAAGCCATTGTTGATTTACTCTTC
AAAACAAATCGAAAAGTAACCGTTAAGCAATTAAAAGAAGATTATTT
CAAAAAAATAGAATGTTTTGATAGTGTTGAAATTTCAGGAGTTGAAG
ATAGATTTAATGCTTCATTAGGTACCTACCATGATTTGCTAAAAATT
ATTAAAGATAAAGATTTTTTGGATAATGAAGAAAATGAAGATATCTT
AGAGGATATTGTTTTAACATTGACCTTATTTGAAGATAGGGAGATGA
TTGAGGAAAGACTTAAAACATATGCTCACCTCTTTGATGATAAGGTG
ATGAAACAGCTTAAACGTCGCCGTTATACTGGTTGGGGACGTTTGTC
TCGAAAATTGATTAATGGTATTAGGGATAAGCAATCTGGCAAAACAA
TATTAGATTTTTTGAAATCAGATGGTTTTGCCAATCGCAATTTTATG
CAGCTGATCCATGATGATAGTTTGACATTTAAAGAAGACATTCAAAA
AGCACAAGTGTCTGGACAAGGCGATAGTTTACATGAACATATTGCAA
ATTTAGCTGGTAGCCCTGCTATTAAAAAAGGTATTTTACAGACTGTA
AAAGTTGTTGATGAATTGGTCAAAGTAATGGGGCGGCATAAGCCAGA
AAATATCGTTATTGAAATGGCACGTGAAAATCAGACAACTCAAAAGG
GCCAGAAAAATTCGCGAGAGCGTATGAAACGAATCGAAGAAGGTATC
AAAGAATTAGGAAGTCAGATTCTTAAAGAGCATCCTGTTGAAAATAC
TCAATTGCAAAATGAAAAGCTCTATCTCTATTATCTCCAAAATGGAA
GAGACATGTATGTGGACCAAGAATTAGATATTAATCGTTTAAGTGAT
TATGATGTCGATCACATTGTTCCACAAAGTTTCCTTAAAGACGATTC
AATAGACAATAAGGTCTTAACGCGTTCTGATAAAAATCGTGGTAAAT
CGGATAACGTTCCAAGTGAAGAAGTAGTCAAAAAGATGAAAAACTAT
TGGAGACAACTTCTAAACGCCAAGTTAATCACTCAACGTAAGTTTGA
TAATTTAACGAAAGCTGAACGTGGAGGTTTGAGTGAACTTGATAAAG
CTGGTTTTATCAAACGCCAATTGGTTGAAACTCGCCAAATCACTAAG
CATGTGGCACAAATTTTGGATAGTCGCATGAATACTAAATACGATGA
AAATGATAAACTTATTCGAGAGGTTAAAGTGATTACCTTAAAATCTA
AATTAGTTTCTGACTTCCGAAAAGATTTCCAATTCTATAAAGTACGT
GAGATTAACAATTACCATCATGCCCATGATGCGTATCTAAATGCCGT
CGTTGGAACTGCTTTGATTAAGAAATATCCAAAACTTGAATCGGAGT
TTGTCTATGGTGATTATAAAGTTTATGATGTTCGTAAAATGATTGCT
AAGTCTGAGCAAGAAATAGGCAAAGCAACCGCAAAATATTTCTTTTA
CTCTAATATCATGAACTTCTTCAAAACAGAAATTACACTTGCAAATG
GAGAGATTCGCAAACGCCCTCTAATCGAAACTAATGGGGAAACTGGA
GAAATTGTCTGGGATAAAGGGCGAGATTTTGCCACAGTGCGCAAAGT
ATTGTCCATGCCCCAAGTCAATATTGTCAAGAAAACAGAAGTACAGA
CAGGCGGATTCTCCAAGGAGTCAATTTTACCAAAAAGAAATTCGGAC
AAGCTTATTGCTCGTAAAAAAGACTGGGATCCAAAAAAATATGGTGG
TTTTGATAGTCCAACGGTAGCTTATTCAGTCCTAGTGGTTGCTAAGG
TGGAAAAAGGGAAATCGAAGAAGTTAAAATCCGTTAAAGAGTTACTA
GGGATCACAATTATGGAAAGAAGTTCCTTTGAAAAAAATCCGATTGA
CTTTTTAGAAGCTAAAGGATATAAGGAAGTTAAAAAAGACTTAATCA
TTAAACTACCTAAATATAGTCTTTTTGAGTTAGAAAACGGTCGTAAA
CGGATGCTGGCTAGTGCCGGAGAATTACAAAAAGGAAATGAGCTGGC
TCTGCCAAGCAAATATGTGAATTTTTTATATTTAGCTAGTCATTATG
AAAAGTTGAAGGGTAGTCCAGAAGATAACGAACAAAAACAATTGTTT
GTGGAGCAGCATAAGCATTATTTAGATGAGATTATTGAGCAAATCAG
TGAATTTTCTAAGCGTGTTATTTTAGCAGATGCCAATTTAGATAAAG
TTCTTAGTGCATATAACAAACATAGAGACAAACCAATACGTGAACAA
GCAGAAAATATTATTCATTTATTTACGTTGACGAATCTTGGAGCTCC
CGCTGCTTTTAAATATTTTGATACAACAATTGATCGTAAACGATATA
CGTCTACAAAAGAAGTTTTAGATGCCACTCTTATCCATCAATCCATC
ACTGGTCTTTATGAAACACGCATTGATTTGAGTCAGCTAGGAGGTGA
CTGAATTAATGCGGCTGCAATTTTTTTGGGCGGGGCCGCCCAAAAAA
ATCCTAGCACCCTGCAGCAGTACTGCTTGACCATAAGAACAAAAAAA
CTTCCGATAAAGTTTGGAAGATAAAGCTAAAAGTTCTTATCTTTGCA
GTATGACGGGAATGTACCCCAGGTTTTAGAGCTAGAAATAGCAAGTT
AAAATAAGGCTAGTCCGTTATCAACTTGAAAAAGTGGCACCGAGTCG
GTGCTTTTTTTGAGATCTGTCGACTCTAGAGGATCCCCGGGTACCGA
GCTCGAATTCACTGGCCGTCGTTTTACAACGTCGTGACTGGGAAAAC
CCTGGCGTTACCCAACTTAATCGTACTTGTGCCTGTTCTATTTCCGA
ACCGACCGCTTGTATGAATCCATCAAAATTCGTTTTCTCTATGTTGG
ATTCCTTGTTGCTCATATTGTGATGATAATTTCTACAAATATAGTCA
TTGGTAACTATCTATGAAACTGTTTGATACTTTTATAGTTGATTAAA
CTTGTTCATGGCATTTGCCTTAATATCATCCGCTATGTCAATGTAGG
GTTTCATAGCTTTGTAGTCGCTGTGTCCCGTCCATTTCATGACCACC
TGTGCCGGGATTCCGAGAGCCAGCGCATTGCAGATGAATGTCCTTTT
TCCTGCATGGGTACTGAGCAAAGCGTATTTGGGTGTGACTTCATCAA
TACGTTCATTTCCCTTGTAGTAGGTTTCCCGTACAGGCTCGTTGATT
TCTGCCAGTTCGCCCAGCTCTTTCAGGTAATCGTTCATCTTCTGGTT
GCTGATGACGGGCAGAGCCATGTAATTCTCGAAATGGATGTCCTTGT
ATTTGTCCAGTATGGCTTTGCTGTATTTGTTCAGTTCAATCGTCAGG
CTGTCGGCAGTCTTGACTGTGGTTATTTCGATGTGGTCGGACTTCAC
ATCGCTTCTTTTCAGATTGCGAACATCCGAATACCGCAAACTCGTAA
AGCAGCAGAACAGGAAAACATCACGCACACGTTCCAGGTATTGCTTA
TCCTTGGGTATCTGGTAGTCTTTCAGCTTGTTCAGTTCATCCCAAGT
CAGGAAGATTACTTTTTTCGAGGTGGTTTTCAGTTTCGGTTTGAACG
TATCGTATGCAATGTTCTGATGATGTCCTTTCTTGAAGCTCCAGCGC
AGGAACCATTTGAGGAATCCCATTTGCTTGCCGATGGTGCTGTTTCT
CATATCCTTGGTGTCACGCAGGAAGTTGACGTATTCGTTCAATCCAA
ACTCGTTGAAATAGTTGAACGTTGCATCCTCCTTGAACTCTTTGAGG
TGGTTCCTCACTGCTGCAAATTTTTCATAGGTGGATGCCGTCCAGTT
ATTCTGGTTACCGCACTCTTTTACAAACTCATCGAACACCTCCCAAA
AGCTGACAGGGGCTTCTTCCGGCTGTTCTTCACTGGTGTCTTTCATT
CTCATGTTGAAAGCTTCCTTCAACTGTTGGGTCGTTGGCATGACCTC
CTGCACCTCAAATTCCTTGAAAATATTCTGGATTTCGGCATAGTATT
TCAGCAAGTCCGTATTGATTTCGGCTGCACTTTGCTTTAGCTTGTTG
GTACATCCGTTCTTTACCCGCTGCTTATCTGCATCCCATTTGGCTAC
GTCAATCCGGTAGCCCGTTGTAAACTCGATACGTTGGCTGGCAAAGA
TGACACGCATACGGATGGGTACGTTCTCTACGATTGGCACACCGTTC
TTTTTCCGGCTCTCCAATGCAAAAATGATGTTGCGCTTGATATTCAT
AATTGGGTGCGTTTGAAATTCTACACCCAAATATACACCCAATTATT
GAGATAGCAAAAGACATTTAGAAACATTTACTTTTACTCTATATTGT
AATTTACACTTGATTATCAGTCGTTTGCAGTCTTATGATATTCTGTG
AAAGTATAAGTTCGAGAGCCTGTCTCTCCGCAAAAAACGCTGAAAAT
CAGCAGATTGCAAAACAAACACCCTGTTTTACACCCAAGAATGTAAA
GTCGGCTGTTTTTGTTTTATTTAAGATAATACAACCACTACATAATA
AAAGAGTAGCGATATTAAAAGAATCCGATGAGAAAAGACTAATATTT
ATCTATCCATTCAGTTTGATTTTTCAGGACTTTACATCGTCCTGAAA
GTATTTGTTGGTACCGGTACCGAGGACGCGTAAACATTTACAGTTGC
ATGTGGCCTATTGTTTTTAGCCGTTAAATATTTTATAACTATTAAAT
AGCGATACAAATTGTTCGAAACTAATATTGTTTATATCATATATTCT
CGCATGTTTTAAAGCTTTATTAAATTGATTTTTTGTAAACAGTTTTT
CGTACTCTTTGTTAACCCATTTCATTACAAAAGTTTCATATTTTTTT
CTCTCTTTAAATGCCATTTTTGCTGGCTTTCTTTTTAATACAATTAA
TGTGCTATCCACTTTAGGTTTTGGATGGAAATAATACCTAGGAATTT
TTGCTAATATAGAAATATCTACCTCTGCCATTAACAGCAATGCTAGT
GATCTGTTTGTATCTAATAACATTTTAGCAAAACCATATTCCACTAT
TAAATAACTTATTGTGGCTGAACTTTCAAAAACAATTTTTCGAATTA
TATTTGTGCTTATGTTGTAAGGTATGCTGCCAAATATTTTATATGGA
TTGTGGCTAGGAAATGTAAATTTCAGTATATCATCATTTACTATTTG
ATAGTTAGGATAATTTAAGAGCTTATTACGAGTTACCTCACATAATT
TAGAATCAATTTCTATCGCCGTTACAAAATTACATCTCTTTACCAAT
CCAGCAGTAAAATGACCTTTCCCTGCACCTATTTCAAAGATGTTATC
TTTTTCATCTAAACTTATGCAATTCATTATTTTTTCTATGTGATATT
TTGAAGTAATAAAATTTTGACTATCTTTTATATTTACTTTGTTCATT
ATAACCTCTCCTTAATTTATTGCATCTCTTTTCGAATATTTATGTTT
TTTGAGAAAAGAACGTACTCATGGTTCATCCCGATATGCGTATCGGT
CTGTATATCAGCAACTTTCTATGTGTTTCAACTACAATAGTCATCTA
TTCTCATCTTTCTGAGTCCACCCCCTGCAAAGCCCCTCTTTACGACA
TAAAAATTCGGTCGGAAAAGGTATGCAAAAGATGTTTCTCTCTTTAA
GAGAAACTCTTCGGGATGCAAAAATATGAAAATAACTCCAATTCACC
AAATTATATAGCGACTTTTTTACAAAATGCTAAAATTTGTTGATTTC
CGTCAAGCAATTGTTGAGCAAAAATGTCTTTTACGATAAAATGATAC
CTCAATATCAACTGTTTAGCAAAACGATATTTCTCTTAAAGAGAGAA
ACACCTTTTTGTTCACCAATCCCCGACTTTTAATCCCGCGGCCATGA
TTGAAAAAGGAAGAGTATGAGTATTCAACATTTCCGTGTCGCCCTTA
TTCCCTTTTTTGCGGCATTTTGCCTTCCTGTTTTTGCTCACCCAGAA
ACGCTGGTGAAAGTAAAAGATGCTGAAGATCAGTTGGGTGCACGAGT
GGGTTACATCGAACTGGATCTCAACAGCGGTAAGATCCTTGAGAGTT
TTCGCCCCGAAGAACGTTTTCCAATGATGAGCACTTTTAAAGTTCTG
CTATGTGGCGCGGTATTATCCCGTATTGACGCCGGGCAAGAGCAACT
CGGTCGCCGCATACACTATTCTCAGAATGACTTGGTTGAGTACTCAC
CAGTCACAGAAAAGCATCTTACGGATGGCATGACAGTAAGAGAATTA
TGCAGTGCTGCCATAACCATGAGTGATAACACTGCGGCCAACTTACT
TCTGACAACGATCGGAGGACCGAAGGAGCTAACCGCTTTTTTGCACA
ACATGGGGGATCATGTAACTCGCCTTGATCGTTGGGAACCGGAGCTG
AATGAAGCCATACCAAACGACGAGCGTGACACCACGATGCCTGTAGC
AATGGCAACAACGTTGCGCAAACTATTAACTGGCGAACTACTTACTC
TAGCTTCCCGGCAACAATTAATAGACTGGATGGAGGCGGATAAAGTT
GCAGGACCACTTCTGCGCTCGGCCCTTCCGGCTGGCTGGTTTATTGC
TGATAAATCTGGAGCCGGTGAGCGTGGGTCTCGCGGTATCATTGCAG
CACTGGGGCCAGATGGTAAGCCCTCCCGTATCGTAGTTATCTACACG
ACGGGGAGTCAGGCAACTATGGATGAACGAAATAGACAGATCGCTGA
GATAGGTGCCTCACTGATTAAGCATTGGTAACTGTCAGACCAAGTTT
ACTCATAACGCGTCAATTCGAGGGGGATCAATTCCGTGATAGGTGGG
CTGCCCTTCCTGGTTGGCTTGGTTTCATCAGCCATCCGCTTGCCCTC
ATCTGTTACGCCGGCGGTAGCCGGCCAGCCTCGCAGAGCAGGATTCC
CGTTGAGCACCGCCAGGTGCGAATAAGGGACAGTGAAGAAGGAACAC
CCGCTCGCGGGTGGGCCTACTTCACCTATCCTGCCCGGCTGACGCCG
TTGGATACACCAAGGAAAGTCTACACGAACCCTTTGGCAAAATCCTG
TATATCGTGCGAAAAAGGATGGATATACCGAAAAAATCGCTATAATG
ACCCCGAAGCAGGGTTATGCAGCGGAAAACGGAATTGATCCGGCCAC
GATGCGTCCGGCGTAGAGGATCTGAAGATCAGCAGTTCAACCTGTTG
ATAGTACGTACTAAGCTCTCATGTTTCACGTACTAAGCTCTCATGTT
TAACGTACTAAGCTCTCATGTTTAACGAACTAAACCCTCATGGCTAA
CGTACTAAGCTCTCATGGCTAACGTACTAAGCTCTCATGTTTCACGT
ACTAAGCTCTCATGTTTGAACAATAAAATTAATATAAATCAGCAACT
TAAATAGCCTCTAAGGTTTTAAGTTTTATAAGAAAAAAAAGAATATA
TAAGGCTTTTAAAGCTTTTAAGGTTTAACGGTTGTGGACAACAAGCC
AGGGATGTAACGCACTGAGAAGCCCTTAGAGCCTCTCAAAGCAATTT
TGAGTGACACAGGAACACTTAACGGCTGACATGGGAATTCCCCTCCA
CCGCGGTGG.

### Example 2

### CRISPR Integration on the Chromosome of Bacteroides thetaiotaomicron

The pNBU2.CRISPR plasmids were transformed to E. coli S-17 lambda-pir, followed by delivery to Bacteroides cells via conjugation. In this specific example, the pNBU2-CRISPR plasmid encodes the intN2 tyrosine integrase, which mediates sequence-specific recombination between the attN2 site on pNBU2-CRISPR plasmid and one of two attBT sites located in the 3′ ends of the two tRNA-Ser genes, BT_t70 (attBT2-1) and BT_t71 (attBT2-2), on the chromosome of B. thetaiotaomicron VPI-5482 (Bt in short). Insertion of the pNBU2-CRISPR plasmid inactivates one of the two tRNA-Ser genes, and simultaneous insertion into both BT_t70 and BT_t71 is unlikely because of the essentiality of tRNA-Ser.

In this specific example, three plasm ids were constructed which express a non-targeting control guide RNA (termed ‘M’), a guide RNA targeting tdk_Bt (BT_2275) and susC_Bt (BT_3702) coding sequences in the Bt genome. The tdk gene encodes thymidine kinase, and the susC gene encodes outer membrane protein involved in starch binding in B. thetaiotaomicron. The protospacer sequence for tdk_Bt is 5′-AATTGAGGCATCGGTCCGAA-3′ (SEQ ID NO: 4), and that for susC_Bt is 5′-ATGACGGGAATGTACCCCAG-3′ (SEQ ID NO: 5). In silico analyses of the non-targeting control protospacer sequence (5′-TGATGGAGAGGTGCAAGTAG-3′; SEQ ID NO: 6) against Bacteroides genomes did not result in any significant sequence matches, so no ‘off-target’ activity is expected. The sgRNA scaffold sequence was 5′-GTTTTAGAGCTAGAAATAGCAAGTTAAAATAAGGCTAGTCCGTTATCAAC TTGAAAAAGTGGCACCGAGTCGGTGCTTTTTT-3″ (SEQ ID NO:7). The resulting plasmids are called pNBU2-CRISPR.M, pNBU2-CRISPR.tdk_Bt, and pNBU2-CRISPR.susC_Bt, respectively.

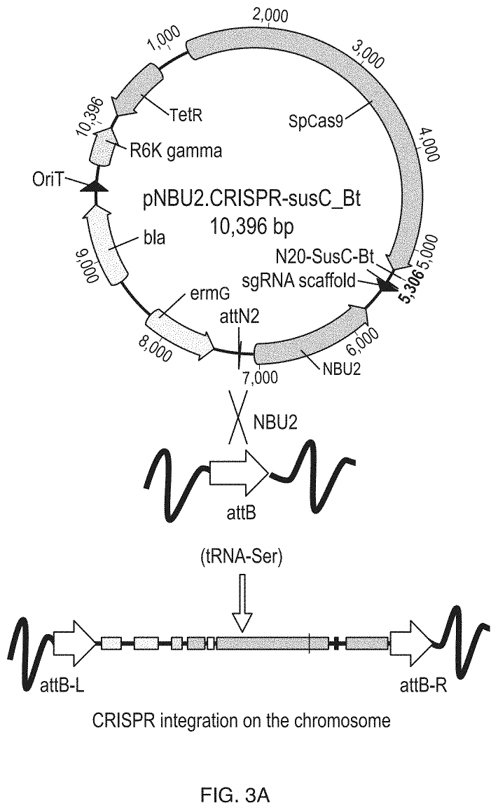

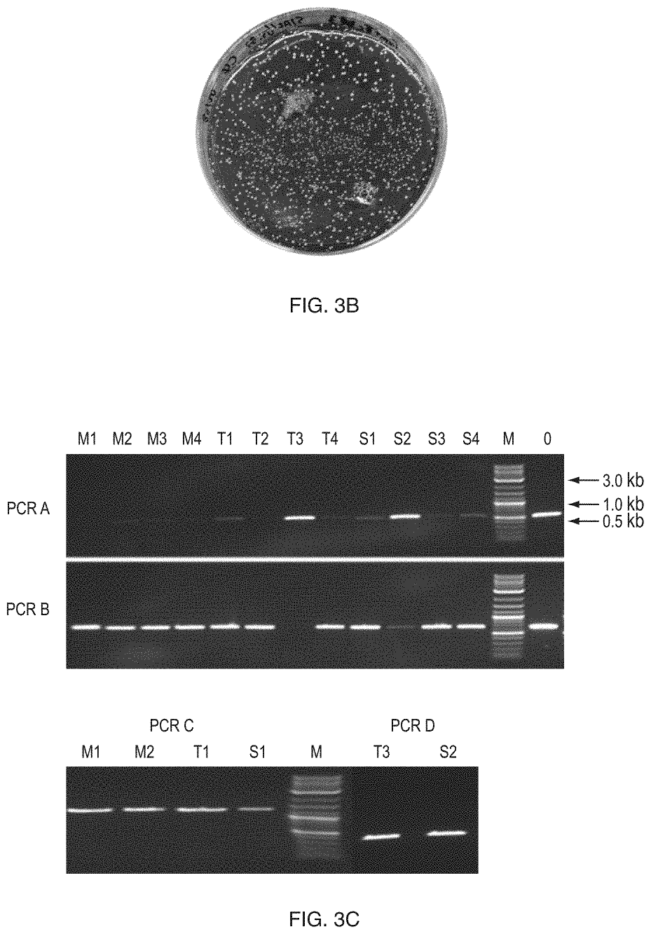

The pNBU2-CRISPR plasmids were conjugated to Bt cells with erythromycin selection, resulting in 500-1000 colonies per conjugation (FIGS. 3A, 3B). Due to a lack of origin of replication for Bacteroides, these plasmids cannot be maintained in Bacteroides cells. The erythromycin resistant colonies were likely chromosomal integrants. Four colonies from each conjugation, labeled as M (M1, M2, M3, M4), tdk_Bt (T1, T2, T3, T4) and susC_Bt (51, S2, S3, S4), were picked for colony PCR screening of CRISPR integration at either one of the two attBT loci (FIG. 3C). Outside primers for each locus were used to identify PCR amplicon sizes: either wild-type or with the plasmid integrated. Since the whole plasmid is about 10 kb, it is unlikely to obtain a PCR amplicon for its integration using colony PCR, while it is possible using purified genomic DNA. For each locus, PCR using outside primers was carried out. If no integration occurred, a PCR amplicon about 0.5 kb (attBT2-1 locus) or 0.65 kb (attBT2-2 locus) is expected on a gel; otherwise, no PCR product is expected. In addition, PCR amplifying left junction of integration was performed using an outside primer binding to chromosomal sequence and an internal primer binding to ermG coding sequence from integration plasmid. If an integration occurred, a PCR product should be seen on gel; otherwise, no PCR product is expected. The PCR amplification was carried out with Q5 Hot-start 2X Master Mix (New England Biolabs), using the following cycling conditions: 98° C. for 30 seconds for initial denaturation; 25 cycles of 98° C. for 20 seconds, 58° C. for 20 seconds, and 72° C. for 45 seconds; and a final extension at 72° C. for 5 minutes. The PCR products were resolved on a 1% agarose gel. As shown in FIG. 3C, based on the sizes of PCR A (attBT2-1 locus) and PCR B (attBT2-2 locus) using the outside primers, it is deduced that clones M1-M4, T1, T2, T4, S1, S3, S4 all harbor CRISPR cassettes integrated at the attBT2-1 locus, while clones T3 and S2 integrated at the attBT2-2 locus on the Bt chromosome. The junctions between chromosome and plasmid sequences were further confirmed correct by PCR (PCR C and D) and Sanger DNA sequencing for selected clones M1, M2, T1, T3, S1 and S2.

### Example 3

### Inducible CRISPR Killing of Individual B. thetaiotaomicron Strains

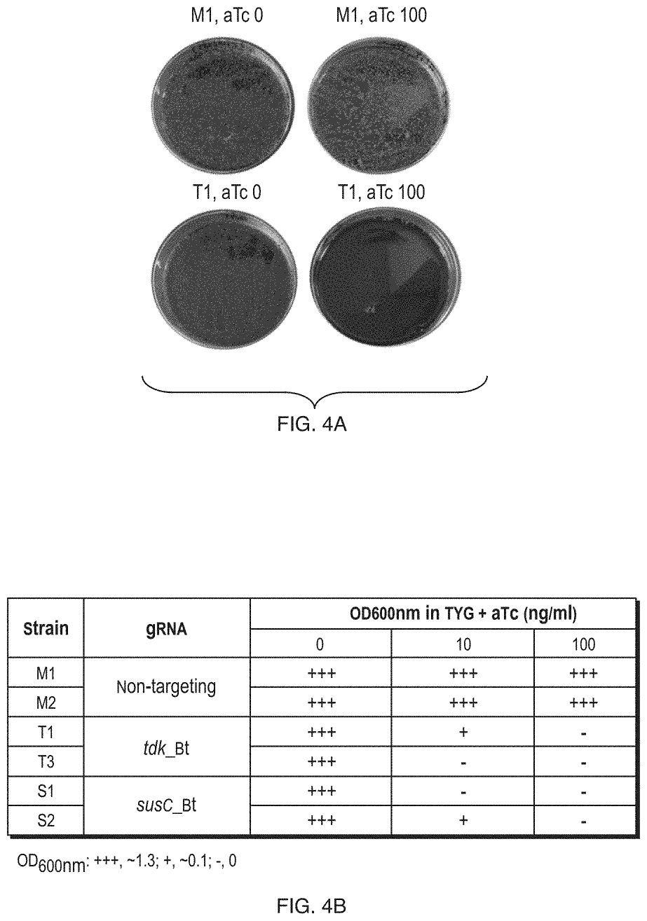

For selected B. thetaiotaomicron CRISPR integrants M1, T1 and S1, all with an inducible CRISPR cassette integrated at the attBT2-1 locus, the inducible CRISPR/Cas9 mediated cell killing was investigated either on BHI blood agar plate or in TYG liquid medium (FIG. 4A and FIG. 4B, respectively). Single colonies of M1 and T1 strains were grown anaerobically in a coy chamber (Coy Laboratory Products Inc.) overnight in falcon tube cultures containing 5 ml TYG liquid medium supplemented with 200 μg/ml gentamicin (Gm) and 25 μg/ml erythromycin (Em). The cultures were diluted (10⁻⁶), and 100 μl were spread onto BHI blood agar plates (Gm 200 μg/ml and Em 25 μg/ml) supplemented with anhydrotetracycline (aTc) at concentrations of 0 and 100 ng/ml, respectively. The agar plates were incubated anaerobically at 37° C. for 2-3 days. About 10³-10⁴ CFU (colony forming units) were obtained on blood agar plates without aTc present (0 ng/ml) for all strains. No CFU formation was observed on blood agar plates with aTc present (100 ng/ml) for the T1strain, while 10³-10⁴ CFU were still obtained for the M1 strain (FIG. 4A).

Similarly, except M1, no cell growth was observed in liquid tube cultures containing TYG medium supplemented with aTc at 100 ng/ml, even after 4 days of anaerobic incubation at 37° C. However, slight growth was observed for clones T1 and S2 at aTc concentration of 10 ng/ml after 24 h of anaerobic incubation, suggesting higher aTc concentration is desired for complete depletion (FIG. 4B). The data shows a chromosomally integrated CRISPR/Cas9 system is activated by exogenously provided inducer aTc to generate lethal genomic DNA cleavages guided by a targeting RNA (tdk_Bt or susC_Bt), resulting in loss of cell viability.

### Example 4

### Targeted, Inducible CRISPR Killing of B. thetaiotaomicron Cells in a Mixed Population In Vitro

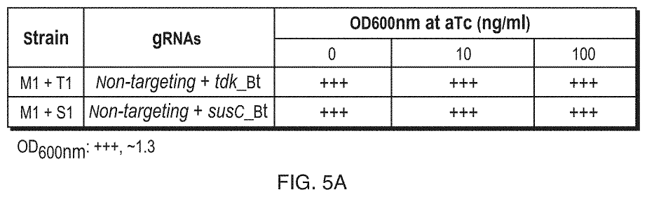

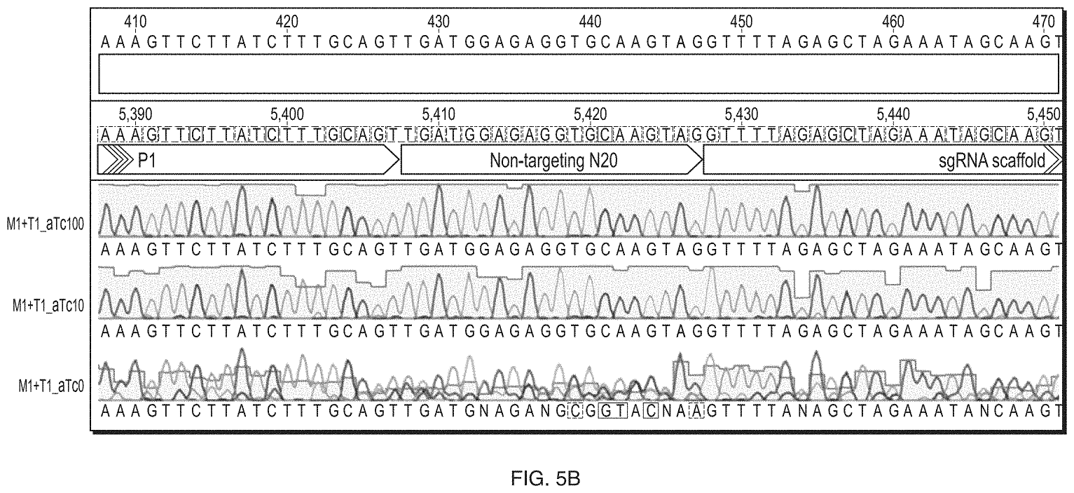

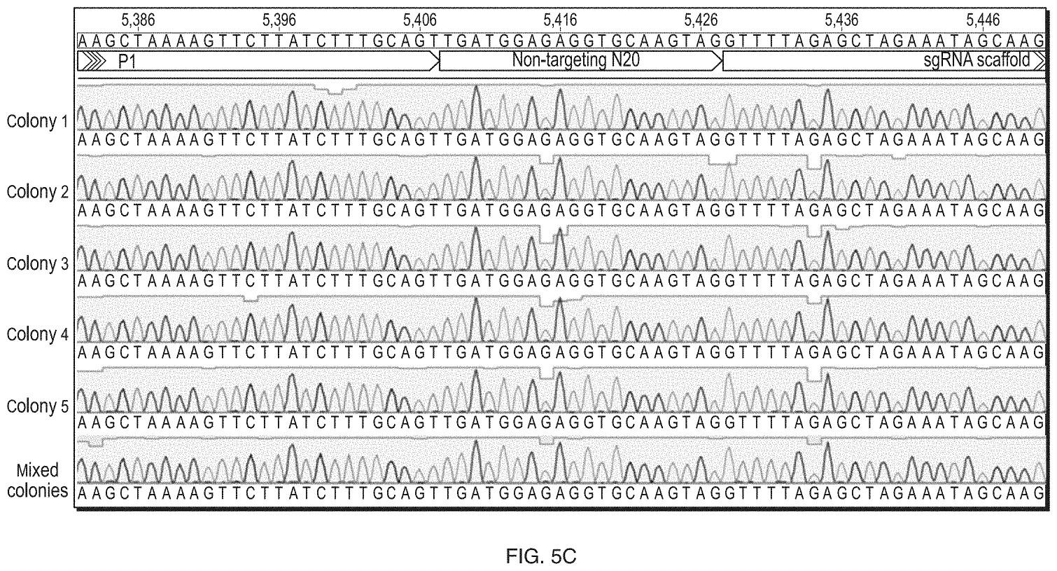

A mixed culture of CRISPR integrated Bt strains expressing either a non-targeting (M) or targeting (tdk_Bt or susC_Bt) guide RNA was employed to demonstrate targeted CRISPR killing of a specific strain in a mixed population in vitro. Equal amounts of exponential growth phase cultures were mixed and incubated anaerobically in 5 ml TYG liquid medium supplemented with aTc at final concentrations of 0, 10 or 100 ng/ml, respectively. After 24 h, all cultures grew up to about 1.3 OD_{600 nm} (FIG. 5A). For one set of the aTc treated cultures (M1+T1, supplemented with aTc at 0, 10 and 100 ng/ml, respectively), PCR and DNA sequencing were performed on the region of guide RNA (P1-N20 sgRNA scaffold). From the DNA sequencing chromatograms, aTc treated cultures (aTc 10 and aTc 100) were only those cells harboring non-targeting, control guide RNA (M), while the culture without aTc treatment (aTc 0) is a mixed population of cells harboring both non-targeting guide RNA (M) and tdk_Bt targeting guide RNA (FIG. 5B).

One of the aTc-treated cultures (M1+S1-aTc100) was diluted and spread on BHI blood agar without aTc supplementation to obtain single colonies. Individual colonies as well as a scrape of colonies on the agar plate were analyzed by PCR of the gRNA region followed by Sanger DNA sequencing. It was found that all the individual colonies and the colony mixture only harbored non-targeting, control gRNA, suggesting that the susC_Bt gRNA harboring integrants were successfully depleted by aTc inducible CRISPR killing, and not growth inhibition due to induced Cas9 protein expression per se in the tube culture (FIG. 5C).

A similar experiment was performed for mixed culture of M1+T1, resulting in the same observations. These data demonstrated targeted, inducible CRISPR killing of B. thetaiotaomicron cells in a mixed population in vitro.

### Example 5

### Long-Term Growth and Targeted CRISPR Killing of B. thetaiotaomicron Strains without Antibiotic Selection

Serial limiting dilution was used for testing long-term growth and targeted killing in liquid cultures without any antibiotic selection. The CRISPR integrated Bt strains M1, T1 and S1 were inoculated in TYG medium from glycerol stocks and grew anaerobically at 37° C. in a coy chamber for 24 h. The culture was re-inoculated into fresh TYG medium at a dilution of 1:100 and grown anaerobically for another 24 h. The same procedures were repeated 4 times, resulting in about 5 days, 40 generations of growth in liquid medium. The cultures were then spread onto BHI blood agar plates forming single colonies. The antibiotic resistances of about 50 colonies each were tested on BHI blood agar plates supplemented with either Gm (200 μg/ml) or Em (25 μg/ml). All colonies tested were resistant to both antibiotics, suggesting the long-term maintenance of the CRISPR cassettes in integrated Bt strains.

### Example 6

### CRISPR Integration on the Chromosome of Bacteroides vulgatus

The inducible CRISPR cassettes were also integrated on the chromosome of Bacteroides vulgatus ATCC 8482 strain (Bv in short). The pNBU2.CRISPR plasmid used for chromosomal integration on Bv was constructed as in Example 1, except that a guide RNA targeting susC_Bv (BVU_RS05095) on Bv genome was cloned. The 20 bp protospacer sequence for expressing susC_Bv guide RNA is 5′-ATTCGGCAGTGAATTCCAGA-3′ (SEQ ID NO: 8).

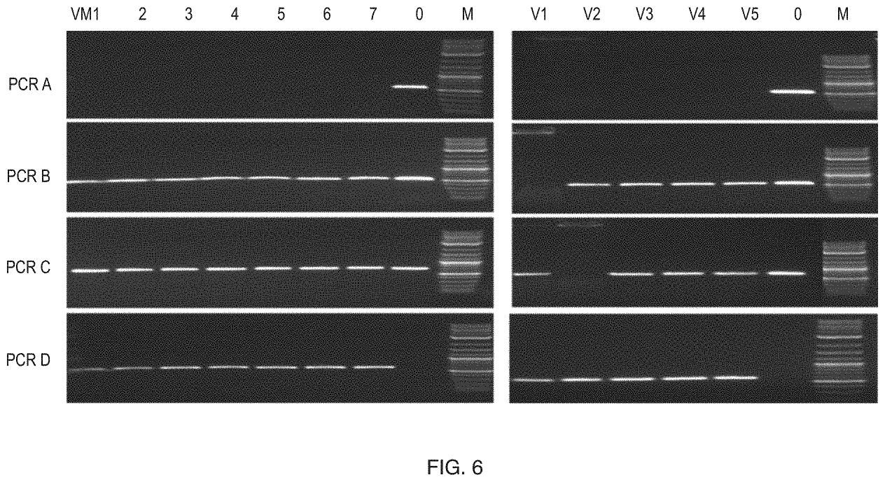

The pNBU2-CRISPR plasmids expressing either non-targeting, control guide RNA (M) or susC_Bv targeting guide RNA were transformed to E. coli S17 lambda-pir, and conjugated to Bv cells. About 10,000 Em resistant colonies were obtained for each conjugation. Seven colonies (labeled VM1, VM2, VM3, VM4, VM5, VM6 and VM7) were picked from the non-targeting control conjugation plate, and five colonies (labeled V1, V2, V3, V4, V5) were picked from the susC_Bv targeting conjugation plate, respectively, for chromosomal CRISPR integration screening by colony PCR. There are three potential NBU2 integrase recognition loci on Bv chromosome, attBv.3-1 (tRNA-Ser, BVU_RS10595), attBv.3-2 (BVU_RS21625) and attBv.3-3 (intergenic region, nucleotide coordinates from 3,171,462 to 3,171,474). For each locus, PCR using outside primers was carried out. If no integration occurred, a PCR amplicon about 0.5 kb is expected on a gel; otherwise, no PCR product is expected. For attBv.3-1 locus, PCR amplifying left junction of integration was performed using an outside primer binding to chromosomal sequence and an internal primer binding to ermG coding sequence from integration plasmid. If an integration occurred at the attBv.3-1 locus, about 0.6 kb PCR product should be seen on a gel; otherwise, no PCR product is expected. As shown in FIG. 6, for non-targeting, control guide RNA integrants (VM), all seven clones harbored CRISPR integration at attBv.3-1 locus; for susC_Bv targeting guide RNA integrants (V), clone V1 may harbor CRISPR integration at both attBv.3-1 and attBv.3-2 loci; clone V2 may harbor CRISPR integration at both attBv.3-1 and attBv.3-3 loci, and for clones V3, V4 and V5, they all harbored CRISPR cassette integration at the attBv.3-1 locus only. This data set shows that the NBU2 based CRISPR integration system works well in Bacteroides vulgatus strains.

### Example 7

### Targeted, Inducible CRISPR Killing of Bacteroides vulgatus

As in Example 3, individual CRISPR integrated By strains VM1 (expressing non-targeting guide RNA) and V1, V2, V3, V4, V5 (all expressing susC_Bv guide RNA) were anaerobically grown in TYG liquid medium overnight. Then the cultures were re-inoculated (1:100 dilution) to fresh TYG medium supplemented with 100 ng/ml aTc, followed by anerobic growth at 37° C. for 24 h. Only VM1 culture grew to high turbidity, while other cultures expressing targeting guide RNA exhibited no growth.

As in Example 4, a mixed culture of VM1 (non-targeting guide RNA) and V3 (expressing susC_Bv guide RNA) were treated with aTc at 100 ng/ml, followed by anaerobic incubation in TYG liquid medium for 24 h. The culture grew up to high turbidity. PCR and DNA sequencing of the guide RNA region of the mixed culture indicates that the treated culture contained only cells expressing non-targeting, control guide RNA. This demonstrates targeted CRISPR killing of specific B. vulgatus strain in a mixed cell population upon the addition of an inducer.

### Example 8

### CRISPR Integration on the Chromosome of Other Bacteroides Strains

The NBU2 integrase recombination tRNA-ser sites (13 bp) are conserved and exist in many other Bacteroides strains as well, including Bacteroides cellulosilyticus, Bacteroides fragilis, Bacteroides helcogenes, Bacteroides ovatus, Bacteroides salanitronis, Bacteroides uniformis and Bacteroides xylanisolvens, based on published genome sequences. The inducible CRISPR cassette expressing a targeting guide RNA can be integrated on the chromosome of these Bacteroides strains (as described in Examples 3 and 6), and targeted CRISPR killing of a specific strain expressing a targeting guide RNA can be achieved by treatment with aTc inducer (as described in Examples 4, 5 and 7).

In situations in which there are no NBU2 integrase sites on the chromosome of a specific species, these 13 base-pair DNA sequences can be readily inserted on the chromosome via recombination (e.g., Cre/loxP) or allelic exchange as described in the art to enable chromosomal CRISPR integration and targeted strain killing.

### Example 9

### CRISPR Integrated Bacteroides Strains Delivered as a Human Probiotic

CRISPR integrated Bacteroides strains can be used as a method to reduce the relative abundance of wildtype Bacteroides strains in the human gut. Anti-PD-1 immunotherapy in human melanoma patients has been shown to differ depending on the presence of Bacteroides strains in gut microbiota (Gopalakrishnan et al., Science, 2018, 359:97-103). Non-responders have increased relative amounts of Bacteroides strains in their microbiota when compared with immunotherapy responders. Before immunotherapy is started, Bacteroides strain elimination, via the induced integrated CRISPR system, could be performed to improve the outcome of human cancer immunotherapy.

### Example 10

### CRISPR-Targeted Reduction in the Representation of a Bacteroides Member of a Model Human Gut Microbiota

The gut microbiota is an important determinant of many aspects of human health as well as various diseases. The rapidly growing appreciation of its myriad effects on host biology has stimulated efforts to develop microbiota-directed therapeutics. Many of these nascent therapies have shown striking dependencies on the initial configuration of the gut microbiota. As such, tools for the delineation of ecological relationships between members of a microbiota (e.g., niche partitioning including the underpinnings of competition/cooperation for nutrients), will play a vital role in the advancement of effective microbiota-directed therapeutics (e.g. Patnode et al., 2019).

In order to study interactions among gut microbial community members, we developed a genetic system with the capability of independently perturbing the representation of specific gut bacterial strains in a defined model human gut microbiota. This system was implemented using Bacteroides thetaiotaomicron, a prominent and common member of the adult human gut microbiota in healthy individuals.

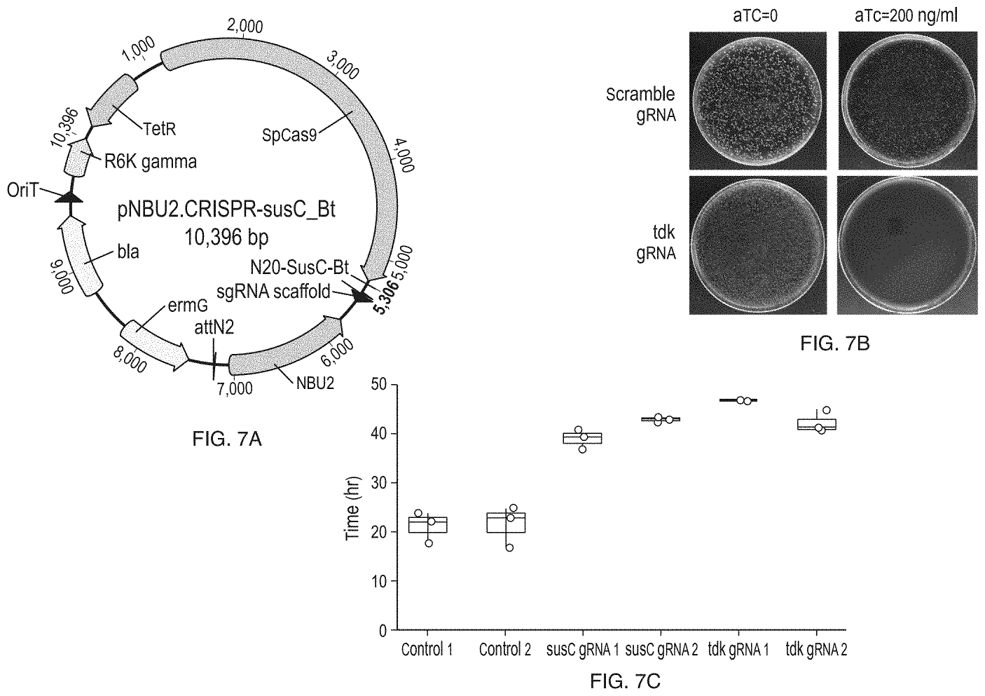

We generated a set of mutants in Bacteroides thetaiotaomicron strain VPI-5482 (Bt). Mutants contained (i) an anhydrotetracycline-inducible (aTc) spCas9 gene, (ii) an erythromycin resistance cassette, and (iii) a constitutively active guide RNA that targets either random, non-genomic sequence (negative control), or one of two Bt genes (tdk and SusC).These cassettes were integrated at one of two genomic locations. Using these mutants, we documented potent aTc-inducible killing in plate assays and in liquid cultures (FIGS. 7A-7C).

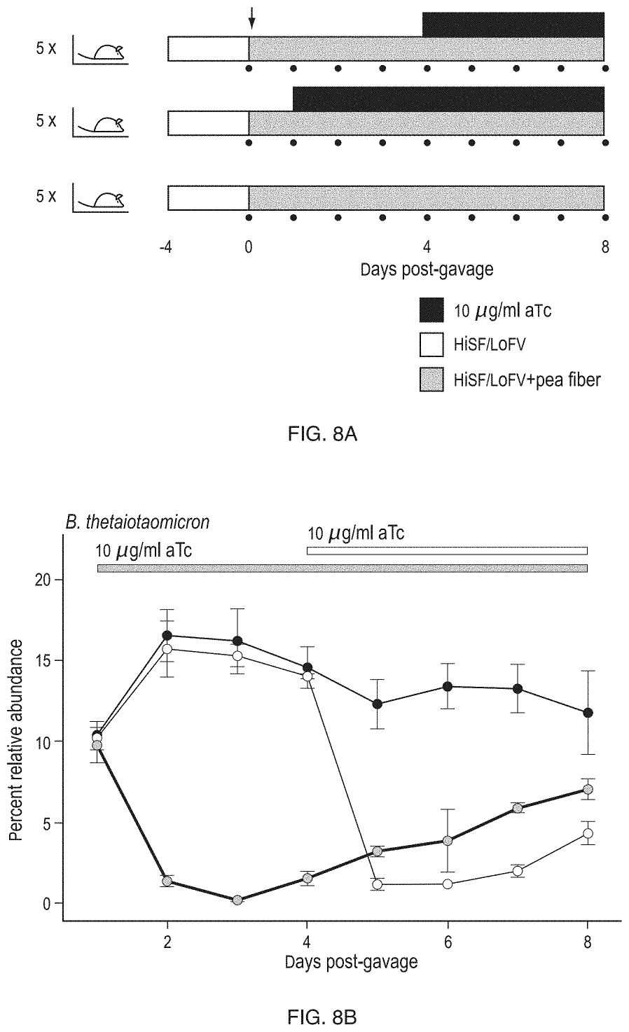

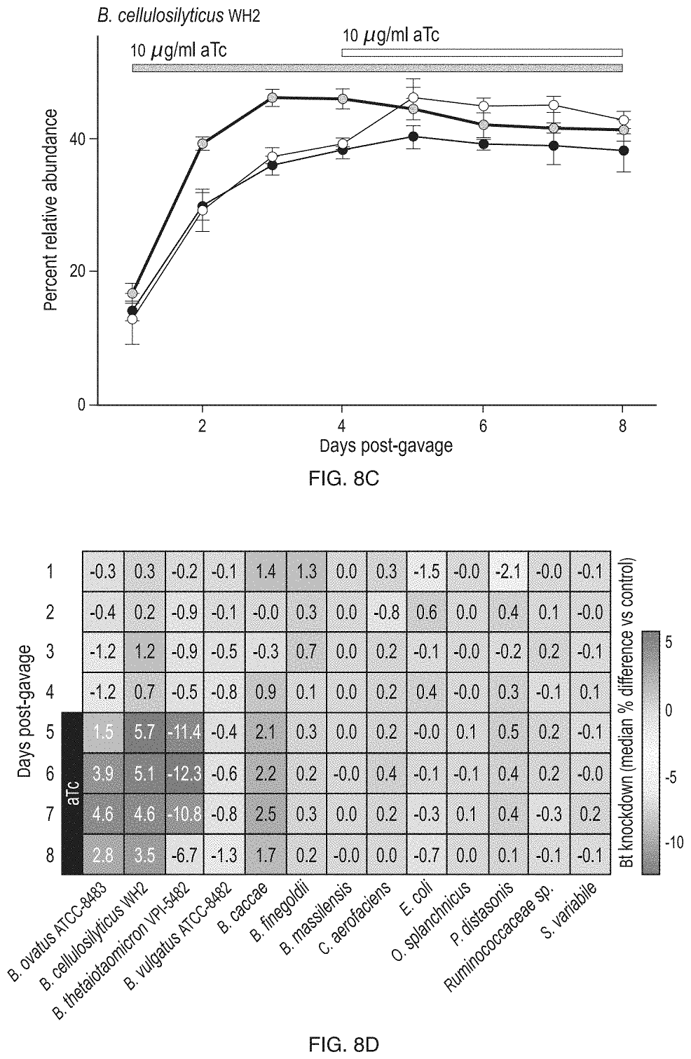

We colonized germ-free mice with a consortium of 13 cultured human gut bacterial strains whose genomes had been sequenced (see below); this consortium included a Bt-CRISPR mutant with a tdk-targeting gRNA. Mice were singly-housed and fed a human diet high in saturated fats and low in fruits and vegetables. The low fiber, ‘HiSF/LoFV’ diet was supplemented with 10% (w/w) pea fiber: this formulation was previously shown to maintain the relative abundance of Bt at 15-20% in this community/diet context (Patnode et al., 2019). In treatment arms, one- or four-days post-gavage, drinking water was supplemented with aTc at 10 μg/mL (FIG. 8A); otherwise after day one post-gavage mice received drinking water containing 0.5% ethanol alone (i.e. the vehicle used to maintain aTc solubility). Short-read shotgun sequencing of fecal DNA was used to define the relative abundances of community members at strain level resolution. In addition, we quantified the absolute abundance of organisms by including two ‘spike-in’ bacterial taxa not found in the mammalian gut (see below for details).

The results revealed that a 35-fold reduction in the relative and in absolute abundances of Bt was achieved 2 days after initiation of aTc treatment in the 4-day treatment arm and had similar effects in the early treatment condition (50-fold relative abundance reduction). Although Bt abundance began increasing after reaching this nadir, it never reached the level observed in the control group (FIG. 8B).

The depletion of Bt was accompanied by significant changes in the relative abundances of several other Bacteroides: B. cellulosilyticus, B. ovatus, and B. caccae (FIG. 8C,D; Linear Mixed Model Marginal Means P<0.05). The pattern of change in absolute abundances of Bt and these other Bacteroides paralleled the relative abundance measurements.

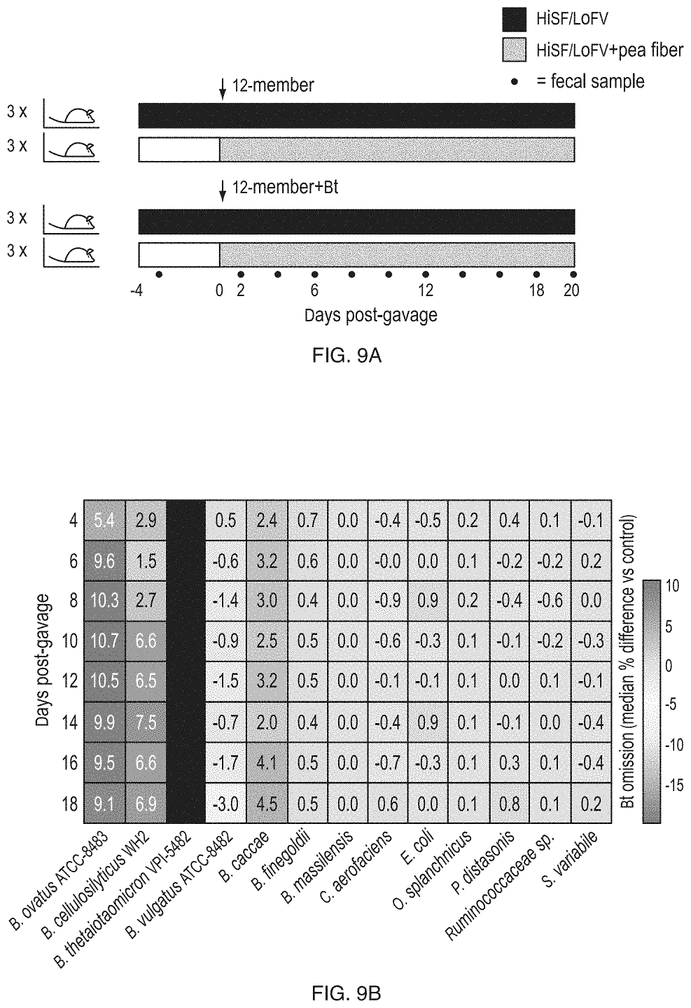

In a related experiment, mice were colonized with the 13-member community, including a WT Bt strain, or a 12-member community, excluding Bt. These mice were singly-housed and fed the HiSF/LoFV+10% pea fiber diet ad libitum for 20 days post-gavage. COPRO-Seq analysis of DNA isolated from serially collected fecal samples disclosed that omission of WT Bt prior to installation of the consortium resulted in changes in the relative/absolute abundances of these other Bacteroides that were largely consistent with the effects of CRISPR-Bt knockdown (FIGS. 9A-9B).

A. In Vitro Growth Assays

All growth assays were performed in a soft-sided anaerobic growth chamber (Coy Laboratory Products) under an atmosphere of 3% hydrogen, 20% CO₂, and 77% N₂. Stock solutions of anhydrotetracycline hydrochloride (37919, Millipore Sigma) were prepared at 2 mg/mL in ethanol and filter-sterilized (Millipore Sigma SLGV033RS). Stocks of Bt mutants were serially diluted and plated on blood agar plates±200 ng/mL anhydrotetracycline. After two days of growth, plates were imaged. Glycerol stocks of Bt mutants were colony purified and inoculated into 5 mL LYBHI containing 25 μg/mL erythromycin. After overnight incubation, cultures were diluted 1:50 in LYBHI medium containing 9 ng/mL aTc and a 200 μL aliquot was pipetted into wells of a 96-well full-area plate (Costar; Cat. No.; CLS3925). Plates were sealed with an optically clear membrane (Axygen; Cat. No.; UC500) and growth monitored via optical density (600 nm) every 15 minutes at 37° C. (Biotek Eon with a BioStack 4).

B. Gnotobiotic Mice

Husbandry—All experiments involving mice were carried out in accordance with protocols approved by the Animal Studies Committee of Washington University in St. Louis. Germ-free male C57/B6 mice (18-22 weeks-old) were singly-housed in cages located within thin-film, flexible plastic isolators and fed an autoclavable mouse chow (Envigo; Cat. No.: 2018S). Cages contained paper houses for environmental enrichment. Animals were maintained on a strict light cycle (lights on at 0600 h, off at 1900 h).

The HiSF/LoFV+10% pea fiber was produced using human foods, selected based on consumption patterns from the National Health and Nutrition Examination Survey (NHANES) database1. The diet was milled to powder (D90 particle size, 980 mm), and mixed with pea fiber at 10% (w/w) fiber (Rattenmaier; Cat. No.: Pea Fiber EF 100). This mixture was then extruded into pellets. The pellets were packaged, vacuum sealed, and sterilized by gamma irradiation (20-50 kilogreys). Sterility was confirmed by culturing the diet under aerobic and anaerobic conditions (atmosphere, 75% N₂, 20% CO₂, 5% H₂) at 37° C. in TYG medium, and by feeding the diets to germ-free mice followed by short read shotgun sequencing (Community PROfiling by sequencing, COPRO-Seq) analysis of their fecal DNA.

Fresh sterile drinking water containing 0.5% ethanol or 10 μg/mL aTC was prepared every other day in gnotobiotic isolators.

Colonization—Bacteroides caccae TSDC17.2-1.2, Bacteroides finegoldii TSDC17.2-1.1, Bacteroides massiliensis TSDC17.2-1.1, Collinsella aerofaciens TSDC17.2-1.1, Escherichia coli TSDC17.2-1.2, Odoribacter splanchnicus TSDC17.2-1.2, Parabacteroides distasonis TSDC17.2-1.1, Ruminococcaceae sp. TSDC17.2-1.2, and Subdoligranulum variabile TSDC17.2-1.1. were cultured from a fecal sample collected from a lean co-twin in an obesity-discordant twin-pair [Twin Pair 1 in Ridaura et al. (2013)]. The annotated genome sequences of these isolates and of Bacteroides ovatus ATCC 8483, Bacteroides vulgatus ATCC 8482, Bacteroides thetaiotaomicron VPI-5482, and Bacteroides cellulosilyticus WH2 are described in Patnode et al 2019. Bacteroides thetaiotaomicron VPI-5482 mutants were generated according to the protocol defined above. Each of these 13 strains were colony purified, grown to early stationary phase in TYGs or LYBHI medium (Goodman et al., 2019). Aliquots of the monocultures were stored at −80° C. in 15% glycerol. Equivalent numbers of organisms were pooled (based on OD600 measurements) and aliquots maintained in 15% glycerol at 80° C. until use. On experimental day 0, aliquots were thawed and introduced into gnotobiotic isolators. The bacterial consortium was administered to germ-free mice through a plastic tipped oral gavage needle (total volume, 300 μL per mouse).

Mice were switched to unsupplemented HiSF/LoFV diet ad libitum for four days prior to colonization. After colonization mice were started on HiSF/LoFV supplemented with 10% pea fiber. One day following gavage, all mice were started on drinking water containing 0.5% ethanol or anhydrotetracycline (10 μg/mL). After colonization and after aTc withdrawal, bedding (Aspen Woodchips; Northeastern Products) was replaced. Fresh fecal samples were collected, within seconds of being produced, from each animal on experimental days 0-8 and immediately frozen at −80° C.

C. COPRO-Seq Analysis of Relative and Absolute Abundances of Community Members

Frozen fecal samples, and cecal contents (collected at the time of euthanasia) were weighed in a 1.8 mL screw-top tube. Two bacteria strains not found in mammalian microbiota were ‘spiked in’ to each sample tube at a known concentration [30 μL of both 2.22×10⁸ cell/mL Alicyclobacillus acidiphilus DSM 14558 and 9.93×10⁸ cell/mL of Agrobacterium radiobacter DSM 30147]. DNA extraction began by bead-beating samples with 250 μL 0.1 mm zirconia/silica beads and one 3.97 mm steel ball in 500 μL of 2× buffer A (200 mM Tris, 200 mM NaCl, 20 mM EDTA), 210 μL 20% (wt:wt) sodium dodecyl sulfate, and 500 μL of phenol:chloroform:amyl alcohol (pH 7.9; 25:24:1) for four minutes (Biospec Minibeadbeater-96). After centrifuging at 3,220 g for 4 minutes, 420 μL of the aqueous phase was removed and purified (QIAquick 96 PCR purification kit; Qiagen) according to the manufacturer's protocol.

Sequencing libraries were prepared from purified DNA using the Nextera DNA Library Prep Kit (Illumina; Cat. No.: 15028211) and combinations of custom barcoded primers (Adey). Libraries were sequenced using an Illumina NextSeq instrument [read length, 75 nt; sequencing depth, 1.02×106±2.2×104 reads/sample (mean±SD)]. Reads were mapped onto bacterial genomes with Bowtie II and relative abundances were calculated using read counts scaled by informative genome size (Hibberd et al., 2017). Samples with less than 100,000 reads were omitted from further analysis. We defined the absolute abundance of given community members using the relationship:

$${ρ}_{species} = \frac{a ( 1 - {P}_{a} - {P}_{r} )}{{P}_{a} W} \cdot {P}_{species}$$

(where Pᵢ is fractional abundance of some species in the 13-member community 1=Σᵢ₌₁¹³ Pᵢ, lower case letters denote cell number, P_{a/r} is the fractional abundance of spike-in bacteria in the total 1=Pₐ+Pᵣ+P_{community}, a denotes A. [symbol] r denotes A. [symbol] , ρ is bacterial density (cells/mg feces), and W is fecal pellet mass (mg)).

D. Linear Modeling

Relative abundance data from the vehicle control arm and the four-day treatment arm for days post gavage 4-7 was modelled using linear models (R core team, 2018) in the form:

lm(log10(percent)˜Condition*DPG, data=data)

Using estimated marginal means (Lenth, 2019), effects of condition by each day were tested for significance on the response scale.

### Example 11

### Vector Construction

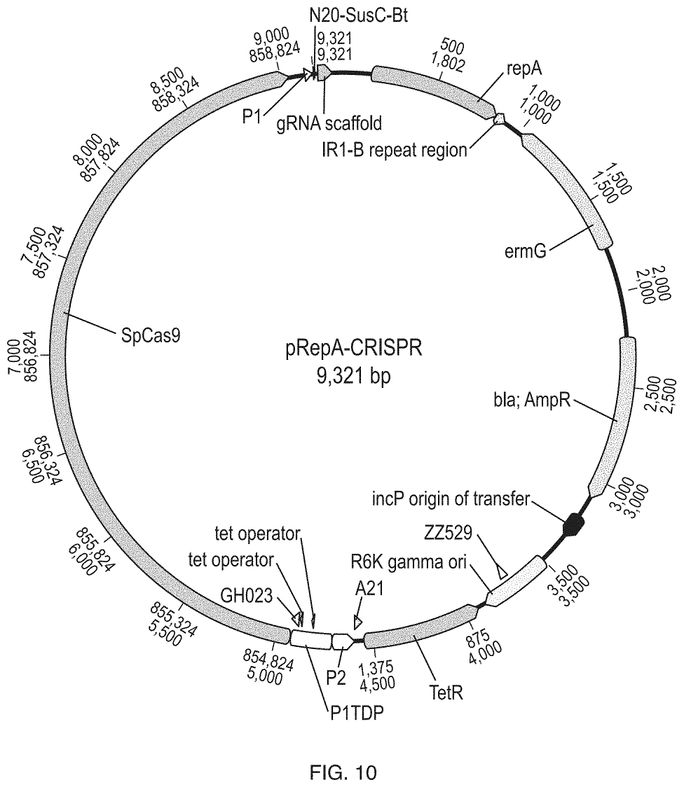

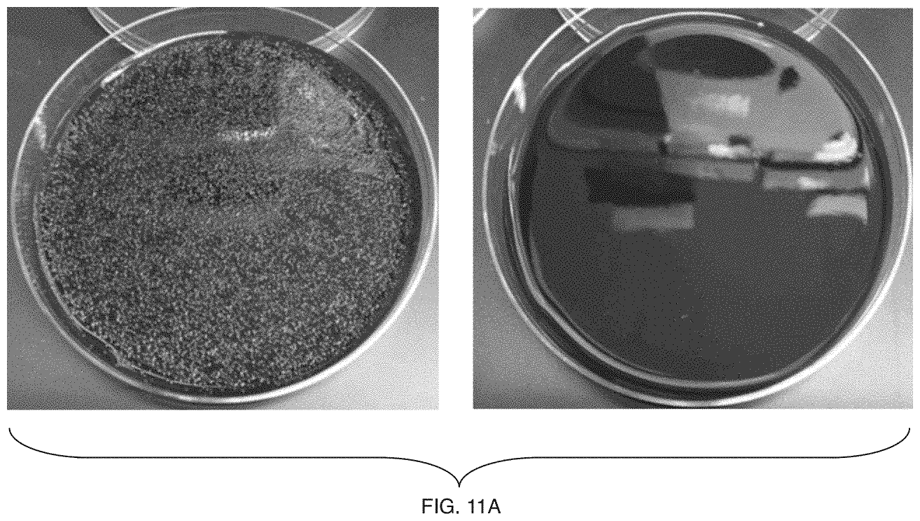

The stably maintained RepA CRISPR plasm ids were constructed using Gibson cloning (NEBuild HIFI DNA Assembly Master Mix, New England Biolabs) of plasmid backbone (RP4-oriT, R6K ori, bla, ermG) from pExchangetdk, RepA from pBI143 (Smith et al., Plasmid, 1995, 34:211-222), and anhydrotetracycline (aTc) inducible CRISPR cassettes (P2-A21-tetR, P1TDP-GH023-SpCas9, P1-N20 sgRNA scaffold) assembled from synthetic DNAs or PCR of genomic DNA of Streptococcus pyogenes strain SF370. FIG. 10 illustrates the plasmid design.

The plasmid backbone harbors R6K origin of replication and bla sequence for ampicillin selection in E. coli, repA sequence for replication in Bacteroides, RP4-oriT sequence for conjugation and ermG sequence for erythromycin (Em) selection in Bacteroides.

The inducible CRISPR cassettes include aTc inducible SpCas9 under the control of TetR regulator (P2-A21-tetR, P1TDP-GH023-SpCas9), and constitutively expressed guide RNA under P1 promoter (P1-N20 sgRNA scaffold). The promoters and ribosomal binding sites are derived and engineered from regulatory sequences of Bacteroides thetaiotaomicron 16S rRNA genes, as described in Lim et al., Cell, 2017, 169:547-558. The guide RNA is a nucleotide sequence that is homologous to a coding DNA sequence, or non-coding DNA sequence, or a non-targeting scramble nucleotide sequence. This sequence can be of any form as long as it is compatible with protospacer adjacent motif (PAM) requirements of different Cas9 homologs. The guide RNA can be either in separate transcriptional units of tracrRNA and crRNA or fused into a hybrid chimeric tracr/crRNA single guide(sgRNA).

The DNA sequence for the above plasmid is presented in SEQ ID NO: 9:

Plasmid (pRepA-CRISPR.susC_Bt)
DNA sequence (9,321 bp)
(SEQ ID NO: 9)
CTCGAGTCCCCGACCGATGATTTTTAAGTGACTGATTTTGTGCT
GTTTTGGGGGTATATTAAGAATAAAAGAAATAGAATAAGTTAAG
TACTTGATACACAATATAGGGCATTTTCCATATTGGAAATTCTC
ATTTTCCAATCTAGAAAATACCGATTTCCTAATATGATACTAAA
TAGGAAAATAATATTTCCCTTAATATTGTTTTTATGGAAAATAA
TAGTTTACTTTGTGGAGAATAATATTTCCCAAAAACATATCAAA
ATGGAAAATAAAAAAGCAGTTAAGTTAACCGATTTTCAAAAGAA
GCAAGAAAATCCTTTTATGAAAACAAGCTATAGAGGTATTGAAA
ATCATGTTGTTAAAAAGTATAAGAGTAATAGTGGTGGCGATAAG
AGAGCTGTAGTAGCTTTAGCCGACACTGAAACTGGAGAAGTGTT
TAAGACTTCGTTTATCCGTCAAATAGAAGTAGATGAAGAACAAT
TCACTAAATTGTATCTTTCTAACTTTGCTGCATTCTTTGACCTA
TCACAAGCAGCTATTCGGGTTTTTGGTTACTTTATGACCTGCAT
GAAACCCAAAAATGATTTAATCATTTTCAATAGAAAAAAATGCC
TAGAATATACCAAATACAAAACAGACAAAGCCGTTTATAAAGGA
CTTGCAGAACTTGTAAAAGCTGAAATCATAGCCCGAGGACCAGC
CGATAATCTTTGGTTTATTAATCCTCTGATAGTATTCAATGGTG
ACCGAGTGACATTTGCTAAAACATACGTTTCGGAAAAAGACTTT
AGCTGCCCAAAAGAAAGAAGAAGCAGAGAAACGACAATTATCAC
TTGGCTTTGATGAACAGTAACACTCCATTGAGTGAAGCTGCCGT
TTGGTCGCTCCCCTTTGGGCGGGGGGGGATAGATAAAGTTCCTC
TATGTAAAGTTATAATGGGGGATGAAGGCAAGGTCGCTAACCTT
ACCGAGGACGCGTAAACATTTACAGTTGCATGTGGCCTATTGTT
TTTAGCCGTTAAATATTTTATAACTATTAAATAGCGATACAAAT
TGTTCGAAACTAATATTGTTTATATCATATATTCTCGCATGTTT
TAAAGCTTTATTAAATTGATTTTTTGTAAACAGTTTTTCGTACT
CTTTGTTAACCCATTTCATTACAAAAGTTTCATATTTTTTTCTC
TCTTTAAATGCCATTTTTGCTGGCTTTCTTTTTAATACAATTAA
TGTGCTATCCACTTTAGGTTTTGGATGGAAATAATACCTAGGAA
TTTTTGCTAATATAGAAATATCTACCTCTGCCATTAACAGCAAT
GCTAGTGATCTGTTTGTATCTAATAACATTTTAGCAAAACCATA
TTCCACTATTAAATAACTTATTGTGGCTGAACTTTCAAAAACAA
TTTTTCGAATTATATTTGTGCTTATGTTGTAAGGTATGCTGCCA
AATATTTTATATGGATTGTGGCTAGGAAATGTAAATTTCAGTAT
ATCATCATTTACTATTTGATAGTTAGGATAATTTAAGAGCTTAT
TACGAGTTACCTCACATAATTTAGAATCAATTTCTATCGCCGTT
ACAAAATTACATCTCTTTACCAATCCAGCAGTAAAATGACCTTT
CCCTGCACCTATTTCAAAGATGTTATCTTTTTCATCTAAACTTA
TGCAATTCATTATTTTTTCTATGTGATATTTTGAAGTAATAAAA
TTTTGACTATCTTTTATATTTACTTTGTTCATTATAACCTCTCC
TTAATTTATTGCATCTCTTTTCGAATATTTATGTTTTTTGAGAA
AAGAACGTACTCATGGTTCATCCCGATATGCGTATCGGTCTGTA
TATCAGCAACTTTCTATGTGTTTCAACTACAATAGTCATCTATT
CTCATCTTTCTGAGTCCACCCCCTGCAAAGCCCCTCTTTACGAC
ATAAAAATTCGGTCGGAAAAGGTATGCAAAAGATGTTTCTCTCT
TTAAGAGAAACTCTTCGGGATGCAAAAATATGAAAATAACTCCA
ATTCACCAAATTATATAGCGACTTTTTTACAAAATGCTAAAATT
TGTTGATTTCCGTCAAGCAATTGTTGAGCAAAAATGTCTTTTAC
GATAAAATGATACCTCAATATCAACTGTTTAGCAAAACGATATT
TCTCTTAAAGAGAGAAACACCTTTTTGTTCACCAATCCCCGACT
TTTAATCCCGCGGCCATGATTGAAAAAGGAAGAGTATGAGTATT
CAACATTTCCGTGTCGCCCTTATTCCCTTTTTTGCGGCATTTTG
CCTTCCTGTTTTTGCTCACCCAGAAACGCTGGTGAAAGTAAAAG
ATGCTGAAGATCAGTTGGGTGCACGAGTGGGTTACATCGAACTG
GATCTCAACAGCGGTAAGATCCTTGAGAGTTTTCGCCCCGAAGA
ACGTTTTCCAATGATGAGCACTTTTAAAGTTCTGCTATGTGGCG
CGGTATTATCCCGTATTGACGCCGGGCAAGAGCAACTCGGTCGC
CGCATACACTATTCTCAGAATGACTTGGTTGAGTACTCACCAGT
CACAGAAAAGCATCTTACGGATGGCATGACAGTAAGAGAATTAT
GCAGTGCTGCCATAACCATGAGTGATAACACTGCGGCCAACTTA
CTTCTGACAACGATCGGAGGACCGAAGGAGCTAACCGCTTTTTT
GCACAACATGGGGGATCATGTAACTCGCCTTGATCGTTGGGAAC
CGGAGCTGAATGAAGCCATACCAAACGACGAGCGTGACACCAC
GATGCCTGTAGCAATGGCAACAACGTTGCGCAAACTATTAACTG
GCGAACTACTTACTCTAGCTTCCCGGCAACAATTAATAGACTGG
ATGGAGGCGGATAAAGTTGCAGGACCACTTCTGCGCTCGGCCC
TTCCGGCTGGCTGGTTTATTGCTGATAAATCTGGAGCCGGTGAG
CGTGGGTCTCGCGGTATCATTGCAGCACTGGGGCCAGATGGTA
AGCCCTCCCGTATCGTAGTTATCTACACGACGGGGAGTCAGGC
AACTATGGATGAACGAAATAGACAGATCGCTGAGATAGGTGCCT
CACTGATTAAGCATTGGTAACTGTCAGACCAAGTTTACTCATAA
CGCGTCAATTCGAGGGGGATCAATTCCGTGATAGGTGGGCTGCC
CTTCCTGGTTGGCTTGGTTTCATCAGCCATCCGCTTGCCCTCAT
CTGTTACGCCGGCGGTAGCCGGCCAGCCTCGCAGAGCAGGAT
TCCCGTTGAGCACCGCCAGGTGCGAATAAGGGACAGTGAAGAA
GGAACACCCGCTCGCGGGTGGGCCTACTTCACCTATCCTGCCC
GGCTGACGCCGTTGGATACACCAAGGAAAGTCTACACGAACCC
TTTGGCAAAATCCTGTATATCGTGCGAAAAAGGATGGATATACC
GAAAAAATCGCTATAATGACCCCGAAGCAGGGTTATGCAGCGG
AAAACGGAATTGATCCGGCCACGATGCGTCCGGCGTAGAGGAT
CTGAAGATCAGCAGTTCAACCTGTTGATAGTACGTACTAAGCTC
TCATGTTTCACGTACTAAGCTCTCATGTTTAACGTACTAAGCTC
TCATGTTTAACGAACTAAACCCTCATGGCTAACGTACTAAGCTC
TCATGGCTAACGTACTAAGCTCTCATGTTTCACGTACTAAGCTC
TCATGTTTGAACAATAAAATTAATATAAATCAGCAACTTAAATA
GCCTCTAAGGTTTTAAGTTTTATAAGAAAAAAAAGAATATATAA
GGCTTTTAAAGCTTTTAAGGTTTAACGGTTGTGGACAACAAGCC
AGGGATGTAACGCACTGAGAAGCCCTTAGAGCCTCTCAAAGCAA
TTTTGAGTGACACAGGAACACTTAACGGCTGACATGGGAATTCC
CCTCCACCGCGGTGGCTTAAGACCCACTTTCACATTTAAGTTGT
TTTTCTAATCCGCATATGATCAATTCAAGGCCGAATAAGAAGGC
TGGCTCTGCACCTTGGTGATCAAATAATTCGATAGCTTGTCGTA
ATAATGGCGGCATACTATCAGTAGTAGGTGTTTCCCTTTCTTCT
TTAGCGACTTGATGCTCTTGATCTTCCAATACGCAACCTAAAGT
AAAATGCCCCACAGCGCTGAGTGCATATAATGCATTCTCTAGTG
AAAAACCTTGTTGGCATAAAAAGGCTAATTGATTTTCGAGAGTT
TCATACTGTTTTTCTGTAGGCCGTGTACCTAAATGTACTTTTGC
TCCATCGCGATGACTTAGTAAAGCACATCTAAAACTTTTAGCGT
TATTACGTAAAAAATCTTGCCAGCTTTCCCCTTCTAAAGGGCAA
AAGTGAGTATGGTGCCTATCTAACATCTCAATGGCTAAGGCGTC
GAGCAAAGCCCGCTTATTTTTTACATGCCAATACAATGTAGGCT
GCTCTACACCTAGCTTCTGGGCGAGTTTACGGGTTGTTAAACCT
TCGATTCCGACCTCATTAAGCAGCTCTAATGCGCTGTTAATCAC
TTTACTTTTATCTAATCTAGACATATTCGTTTAATATCATAAAT
AATTTATTTTATTTTAAAATGCGCGGGTGCAAAGGTAAGAGGTT
TTATTTTAACTACCAAATGTTTTCGGAAGTTTTTTCGCTTTTCT
TTTTCTATCGTTTCTCAGACTCTCTTAGCGAAAGGGAAAGAAGG
TAAAGAAGAAAAACAAAACGCCTTTTCTTTTTTGCACCCGCTTT
CCAAGAGAAGAAAGCCTTGTTAAATTGACTTAGTGTAAAAGCGC
AGTACTGCTTGACCATAAGAACAAAAAAATCTCTATCACTGATA
GGGATAAAGTTTGGAAGATAAAGCTAAAAGTTCTTATCTTTGCA
GTCTCCCTATCAGTGATAGAGACGAAATAAAGACATATAAAAGA
AAAGACACCATGGATAAGAAATACTCAATAGGCTTAGATATCGG
CACAAATAGCGTCGGATGGGCGGTGATCACTGATGAATATAAGG
TTCCGTCTAAAAAGTTCAAGGTTCTGGGAAATACAGACCGCCAC
AGTATCAAAAAAAATCTTATAGGGGCTCTTTTATTTGACAGTGG
AGAGACAGCGGAAGCGACTCGTCTCAAACGGACAGCTCGTAGAA
GGTATACACGTCGGAAGAATCGTATTTGTTATCTACAGGAGATT
TTTTCAAATGAGATGGCGAAAGTAGATGATAGTTTCTTTCATCG
ACTTGAAGAGTCTTTTTTGGTGGAAGAAGACAAGAAGCATGAAC
GTCATCCTATTTTTGGAAATATAGTAGATGAAGTTGCTTATCAT
GAGAAATATCCAACTATCTATCATCTGCGAAAAAAATTGGTAGA
TTCTACTGATAAAGCGGATTTGCGCTTAATCTATTTGGCCTTAG
CGCATATGATTAAGTTTCGTGGTCATTTTTTGATTGAGGGAGAT
TTAAATCCTGATAATAGTGATGTGGACAAACTATTTATCCAGTT
GGTACAAACCTACAATCAATTATTTGAAGAAAACCCTATTAACG
CAAGTGGAGTAGATGCTAAAGCGATTCTTTCTGCACGATTGAGT
AAATCAAGACGATTAGAAAATCTCATTGCTCAGCTCCCCGGTGA
GAAGAAAAATGGCTTATTTGGGAATCTCATTGCTTTGTCATTGG
GTTTGACCCCTAATTTTAAATCAAATTTTGATTTGGCAGAAGAT
GCTAAATTACAGCTTTCAAAAGATACTTACGATGATGATTTAGA
TAATTTATTGGCGCAAATTGGAGATCAATATGCTGATTTGTTTT
TGGCAGCTAAGAATTTATCAGATGCTATTTTACTTTCAGATATC
CTAAGAGTAAATACTGAAATAACTAAGGCTCCCCTATCAGCTTC
AATGATTAAACGCTACGATGAACATCATCAAGACTTGACTCTTT
TAAAAGCTTTAGTTCGACAACAACTTCCAGAAAAGTATAAAGAA
ATCTTTTTTGATCAATCAAAAAACGGATATGCAGGTTATATTGA
TGGGGGAGCTAGCCAAGAAGAATTTTATAAATTTATCAAACCAA
TTTTAGAAAAAATGGATGGTACTGAGGAATTATTGGTGAAACTA
AATCGTGAAGATTTGCTGCGCAAGCAACGGACCTTTGACAACGG
CTCTATTCCCCATCAAATTCACTTGGGTGAGCTGCATGCTATTT
TGAGAAGACAAGAAGACTTTTATCCATTTTTAAAAGACAATCGT
GAGAAGATTGAAAAAATCTTGACTTTTCGAATTCCTTATTATGT
TGGTCCATTGGCGCGTGGCAATAGTCGTTTTGCATGGATGACTC
GGAAGTCTGAAGAAACAATTACCCCATGGAATTTTGAAGAAGTT
GTCGATAAAGGTGCTTCAGCTCAATCATTTATTGAACGCATGAC
AAACTTTGATAAAAATCTTCCAAATGAAAAAGTACTACCAAAAC
ATAGTTTGCTTTATGAGTATTTTACGGTTTATAACGAATTGACA
AAGGTCAAATATGTTACTGAAGGAATGCGAAAACCAGCATTTCT
TTCAGGTGAACAGAAGAAAGCCATTGTTGATTTACTCTTCAAAA
CAAATCGAAAAGTAACCGTTAAGCAATTAAAAGAAGATTATTTC
AAAAAAATAGAATGTTTTGATAGTGTTGAAATTTCAGGAGTTGA
AGATAGATTTAATGCTTCATTAGGTACCTACCATGATTTGCTAA
AAATTATTAAAGATAAAGATTTTTTGGATAATGAAGAAAATGAA
GATATCTTAGAGGATATTGTTTTAACATTGACCTTATTTGAAGA
TAGGGAGATGATTGAGGAAAGACTTAAAACATATGCTCACCTCT
TTGATGATAAGGTGATGAAACAGCTTAAACGTCGCCGTTATACT
GGTTGGGGACGTTTGTCTCGAAAATTGATTAATGGTATTAGGGA
TAAGCAATCTGGCAAAACAATATTAGATTTTTTGAAATCAGATG
GTTTTGCCAATCGCAATTTTATGCAGCTGATCCATGATGATAGT
TTGACATTTAAAGAAGACATTCAAAAAGCACAAGTGTCTGGACA
AGGCGATAGTTTACATGAACATATTGCAAATTTAGCTGGTAGCC
CTGCTATTAAAAAAGGTATTTTACAGACTGTAAAAGTTGTTGAT
GAATTGGTCAAAGTAATGGGGCGGCATAAGCCAGAAAATATCGT
TATTGAAATGGCACGTGAAAATCAGACAACTCAAAAGGGCCAGA
AAAATTCGCGAGAGCGTATGAAACGAATCGAAGAAGGTATCAAA
GAATTAGGAAGTCAGATTCTTAAAGAGCATCCTGTTGAAAATAC
TCAATTGCAAAATGAAAAGCTCTATCTCTATTATCTCCAAAATG
GAAGAGACATGTATGTGGACCAAGAATTAGATATTAATCGTTTA
AGTGATTATGATGTCGATCACATTGTTCCACAAAGTTTCCTTAA
AGACGATTCAATAGACAATAAGGTCTTAACGCGTTCTGATAAAA
ATCGTGGTAAATCGGATAACGTTCCAAGTGAAGAAGTAGTCAAA
AAGATGAAAAACTATTGGAGACAACTTCTAAACGCCAAGTTAAT
CACTCAACGTAAGTTTGATAATTTAACGAAAGCTGAACGTGGAG
GTTTGAGTGAACTTGATAAAGCTGGTTTTATCAAACGCCAATTG
GTTGAAACTCGCCAAATCACTAAGCATGTGGCACAAATTTTGGA
TAGTCGCATGAATACTAAATACGATGAAAATGATAAACTTATTC
GAGAGGTTAAAGTGATTACCTTAAAATCTAAATTAGTTTCTGAC
TTCCGAAAAGATTTCCAATTCTATAAAGTACGTGAGATTAACAA
TTACCATCATGCCCATGATGCGTATCTAAATGCCGTCGTTGGAA
CTGCTTTGATTAAGAAATATCCAAAACTTGAATCGGAGTTTGTC
TATGGTGATTATAAAGTTTATGATGTTCGTAAAATGATTGCTAA
GTCTGAGCAAGAAATAGGCAAAGCAACCGCAAAATATTTCTTTT
ACTCTAATATCATGAACTTCTTCAAAACAGAAATTACACTTGCA
AATGGAGAGATTCGCAAACGCCCTCTAATCGAAACTAATGGGGA
AACTGGAGAAATTGTCTGGGATAAAGGGCGAGATTTTGCCACAG
TGCGCAAAGTATTGTCCATGCCCCAAGTCAATATTGTCAAGAAA
ACAGAAGTACAGACAGGCGGATTCTCCAAGGAGTCAATTTTACC
AAAAAGAAATTCGGACAAGCTTATTGCTCGTAAAAAAGACTGGG
ATCCAAAAAAATATGGTGGTTTTGATAGTCCAACGGTAGCTTAT
TCAGTCCTAGTGGTTGCTAAGGTGGAAAAAGGGAAATCGAAGAA
GTTAAAATCCGTTAAAGAGTTACTAGGGATCACAATTATGGAAA
GAAGTTCCTTTGAAAAAAATCCGATTGACTTTTTAGAAGCTAAA
GGATATAAGGAAGTTAAAAAAGACTTAATCATTAAACTACCTAA
ATATAGTCTTTTTGAGTTAGAAAACGGTCGTAAACGGATGCTGG
CTAGTGCCGGAGAATTACAAAAAGGAAATGAGCTGGCTCTGCCA
AGCAAATATGTGAATTTTTTATATTTAGCTAGTCATTATGAAAA
GTTGAAGGGTAGTCCAGAAGATAACGAACAAAAACAATTGTTTG
TGGAGCAGCATAAGCATTATTTAGATGAGATTATTGAGCAAATC
AGTGAATTTTCTAAGCGTGTTATTTTAGCAGATGCCAATTTAGA
TAAAGTTCTTAGTGCATATAACAAACATAGAGACAAACCAATAC
GTGAACAAGCAGAAAATATTATTCATTTATTTACGTTGACGAAT
CTTGGAGCTCCCGCTGCTTTTAAATATTTTGATACAACAATTGA
TCGTAAACGATATACGTCTACAAAAGAAGTTTTAGATGCCACTC
TTATCCATCAATCCATCACTGGTCTTTATGAAACACGCATTGAT
TTGAGTCAGCTAGGAGGTGACTGAATTAATGCGGCTGCAATTTT
TTTGGGCGGGGCCGCCCAAAAAAATCCTAGCACCCTGCAGCAGT
ACTGCTTGACCATAAGAACAAAAAAACTTCCGATAAAGTTTGGA
AGATAAAGCTAAAAGTTCTTATCTTTGCAGTATGACGGGAATGT
ACCCCAGGTTTTAGAGCTAGAAATAGCAAGTTAAAATAAGGCTA
GTCCGTTATCAACTTGAAAAAGTGGCACCGAGTCGGTGCTTTTT
TTGAGATCTGTCCATACCCATGGGACGTCTGATGATTAAGGATC
TTGC

### Example 12

### Inducible CRISPR Killing of Individual B. thetaiotaomicron Strains

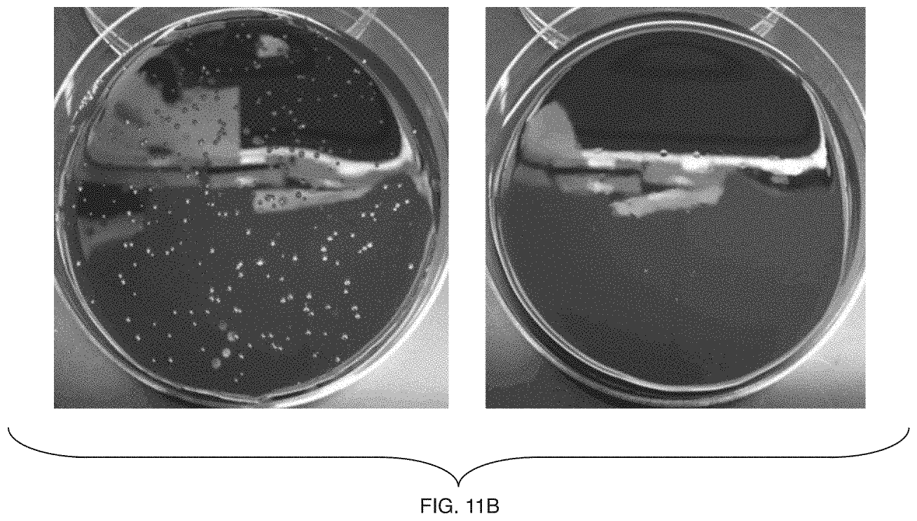

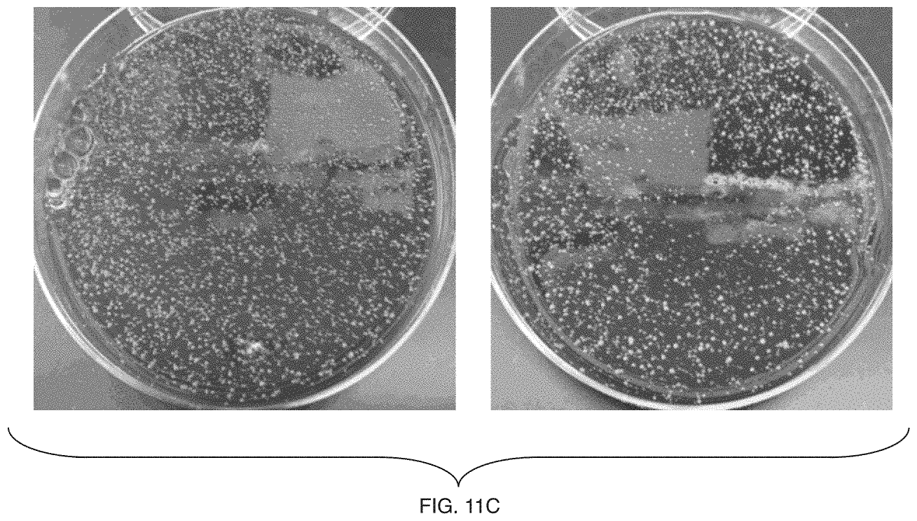

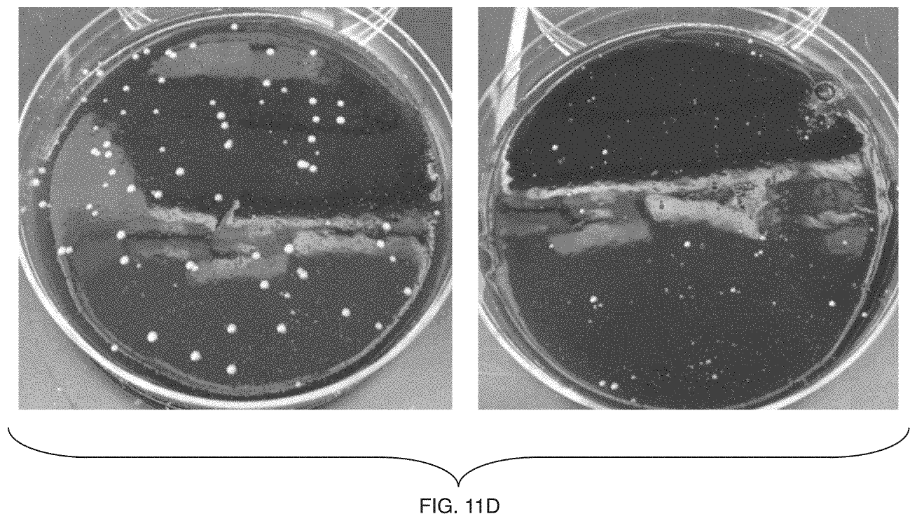

Two stably maintained plasmids were conjugated into B. thetaiotaomicron on Brain Heart Infusion (BHI) blood agar plates with no antibiotic selection. One plasmid was a negative control (termed ‘M’) with a scrambled non-targeting protospacer sequence (5′-TGATGGAGAGGTGCAAGTAG-3′; SEQ ID NO 6). In silico analyses of this non-targeting control protospacer sequence against Bacteroides genomes did not result in any significant sequence matches, so no ‘off-target’ activity is expected. The other plasmid has a protospacer sequence (5′-ATGACGGGAATGTACCCCAG-3′; SEQ ID NO:5) that targets the susC_Bt (BT_3702) coding sequence on the Bt genome. susC gene encodes an outer membrane protein involved in starch binding in B. thetaiotaomicron. The sgRNA scaffold sequence was 5′-GTTTTAGAGCTAGAAATAGCAAGTTAAAATAAGGCTAGTCCGTTATCAAC TTGAAAA AGTGGCACCGAGTCGGTGCTTTTTT-3′ (SEQ ID NO:7). The resulting two plasm ids are called pRepA-CRISPR.Mand pRepA-CRISPR.susC_Bt. Colonies were picked and re-streaked on BHI blood agar plates with 200 μg/ml gentamin (Gm) and 50 μg/ml erythromycin (Em) and grown anaerobically in a coy chamber (Coy Laboratory Products Inc.). From this re-streaked plate, a single colony was picked and grown in 10 ml of TYG liquid medium at 200 μg/ml Gm and 50 μg/ml Em. OD_{600 nm} readings were taken so the concentrations could be adjusted to an OD of 1. One to ten dilutions were then made in one ml volumes. One hundred microliters of two dilutions (10₋₄ and 10₋₆) were spread on BHI blood agar plates with 200 μg/ml gentamin (Gm) and 50 μg/ml erythromycin (Em) supplemented with anhydrotetracycline (aTc) at concentrations of 0 and 100 ng/ml, respectively. The agar plates were incubated anaerobically at 37° C. for 2-3 days. Colony forming units (CFU) were obtained on blood agar plates without aTc present (0 ng/ml) for all strains. No CFU formation was observed on blood agar plates with aTc present (100 ng/ml) for the susC targeting plasmid, while CFUs were still obtained for the M strain (FIGS. 11 A-D).

### Example 13

### Vector Construction

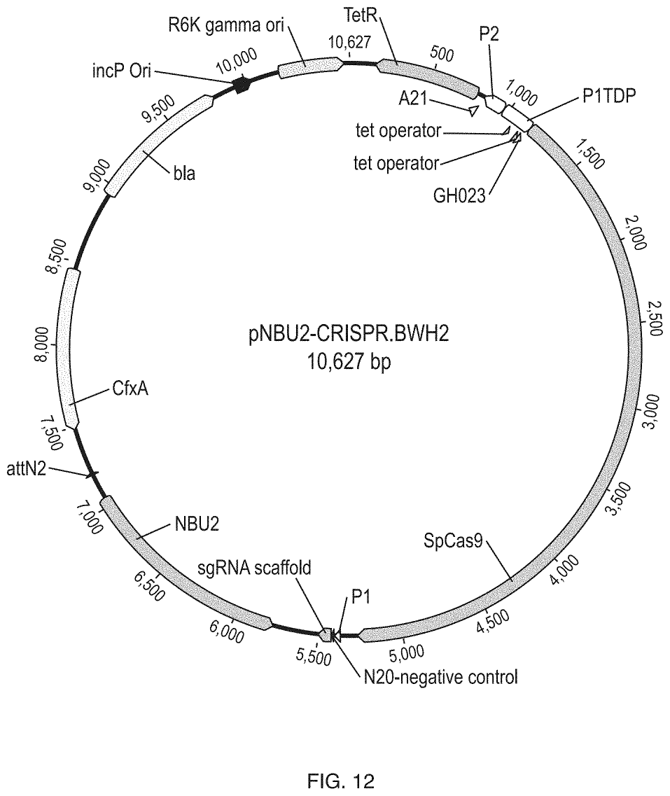

The CRISPR integration pNBU2.CRISPR plasm ids were constructed using Gibson cloning (NEBuild HIFI DNA Assembly Master Mix, New England Biolabs) of plasmid backbone (RP4-oriT, R6K ori, bla, ermG) from pExchangetdk, NBU2 integrase from pNBU2-tetQb, and an anhydrotetracycline (aTc) inducible CRISPR cassettes (P2-A21-tetR, P1TDP-GH023-SpCas9, P1-N20 sgRNA scaffold) assembled from synthetic DNAs or PCR of genomic DNA of Streptococcus pyogenes strain SF370. The erythromycin (ermG) antibiotic resistance gene was replaced by the cefoxitin antibiotic resistance gene (cfxA) using synthetic DNA and traditional restriction enzyme cloning. FIG. 12 illustrates the plasmid design.

The plasmid backbone harbors R6K origin of replication and bla sequence for ampicillin selection in E. coli, RP4-oriT sequence for conjugation and cfxA sequence for cefoxitin (FOX) selection in Bacteroides (Parker and Smith, Antimicrobial agents and Chemotherapy, 1993, 37: 1028-1036). NBU2 encodes the intN2 tyrosine integrase, which mediates sequence-specific recombination between the attN2 site on pNBU2-CRISPR plasmid and one of the attB sites located on the chromosome of Bacteroides cells. The attN2 and attB have the same 13 bp recognition nucleotide sequence (5′-3′): CCTGTCTCTCCGC (SEQ ID NO: 2).

The inducible CRISPR cassettes include aTc inducible SpCas9 under the control of TetR regulator (P2-A21-tetR, P1TDP-GH023-SpCas9), and constitutively expressed guide RNA under P1 promoter (P1-N20 sgRNA scaffold). The promoters and ribosomal binding sites are derived and engineered from regulatory sequences of Bacteroides thetaiotaomicron 16S rRNA genes, as described in Lim et al., Cell, 2017, 169:547-558. The guide RNA is a nucleotide sequence that is homologous to a coding DNA sequence, or non-coding DNA sequence, or a non-targeting scramble nucleotide sequence. This sequence can be of any form as long as it is compatible with protospacer adjacent motif (PAM) requirements of different Cas9 homologs. The guide RNA can be either in separate transcriptional units of tracrRNA and crRNA or fused into a hybrid chimeric tracr/crRNA single guide(sgRNA).

The DNA sequence for the above plasmid (FIG. 12) is presented in SEQ ID NO:10:

Plasmid (pNBU2-CRISPR.non-targeting M_BWH2)
DNA sequence (10,627 bp)
(SEQ ID NO: 10)
GGAAAGCGGGCAGTGAGCGCAACGCAATTAATGTGAGTTAGCTCACT
CATTAGGCACCCCAGGCTTTACACTTTATGCTTCCGGCTCGTATGTT
GTGTGGAATTGTGAGCGGATAACAATTTCACACAGGAAACAGCTATG
ACCATGATTACGCCCTTAAGACCCACTTTCACATTTAAGTTGTTTTT
CTAATCCGCATATGATCAATTCAAGGCCGAATAAGAAGGCTGGCTCT
GCACCTTGGTGATCAAATAATTCGATAGCTTGTCGTAATAATGGCGG
CATACTATCAGTAGTAGGTGTTTCCCTTTCTTCTTTAGCGACTTGAT
GCTCTTGATCTTCCAATACGCAACCTAAAGTAAAATGCCCCACAGCG
CTGAGTGCATATAATGCATTCTCTAGTGAAAAACCTTGTTGGCATAA
AAAGGCTAATTGATTTTCGAGAGTTTCATACTGTTTTTCTGTAGGCC
GTGTACCTAAATGTACTTTTGCTCCATCGCGATGACTTAGTAAAGCA
CATCTAAAACTTTTAGCGTTATTACGTAAAAAATCTTGCCAGCTTTC
CCCTTCTAAAGGGCAAAAGTGAGTATGGTGCCTATCTAACATCTCAA
TGGCTAAGGCGTCGAGCAAAGCCCGCTTATTTTTTACATGCCAATAC
AATGTAGGCTGCTCTACACCTAGCTTCTGGGCGAGTTTACGGGTTGT
TAAACCTTCGATTCCGACCTCATTAAGCAGCTCTAATGCGCTGTTAA
TCACTTTACTTTTATCTAATCTAGACATATTCGTTTAATATCATAAA
TAATTTATTTTATTTTAAAATGCGCGGGTGCAAAGGTAAGAGGTTTT
ATTTTAACTACCAAATGTTTTCGGAAGTTTTTTCGCTTTTCTTTTTC
TATCGTTTCTCAGACTCTCTTAGCGAAAGGGAAAGAAGGTAAAGAAG
AAAAACAAAACGCCTTTTCTTTTTTGCACCCGCTTTCCAAGAGAAGA
AAGCCTTGTTAAATTGACTTAGTGTAAAAGCGCAGTACTGCTTGACC
ATAAGAACAAAAAAATCTCTATCACTGATAGGGATAAAGTTTGGAAG
ATAAAGCTAAAAGTTCTTATCTTTGCAGTCTCCCTATCAGTGATAGA
GACGAAATAAAGACATATAAAAGAAAAGACACCATGGATAAGAAATA
CTCAATAGGCTTAGATATCGGCACAAATAGCGTCGGATGGGCGGTGA
TCACTGATGAATATAAGGTTCCGTCTAAAAAGTTCAAGGTTCTGGGA
AATACAGACCGCCACAGTATCAAAAAAAATCTTATAGGGGCTCTTTT
ATTTGACAGTGGAGAGACAGCGGAAGCGACTCGTCTCAAACGGACAG
CTCGTAGAAGGTATACACGTCGGAAGAATCGTATTTGTTATCTACAG
GAGATTTTTTCAAATGAGATGGCGAAAGTAGATGATAGTTTCTTTCA
TCGACTTGAAGAGTCTTTTTTGGTGGAAGAAGACAAGAAGCATGAAC
GTCATCCTATTTTTGGAAATATAGTAGATGAAGTTGCTTATCATGAG
AAATATCCAACTATCTATCATCTGCGAAAAAAATTGGTAGATTCTAC
TGATAAAGCGGATTTGCGCTTAATCTATTTGGCCTTAGCGCATATGA
TTAAGTTTCGTGGTCATTTTTTGATTGAGGGAGATTTAAATCCTGAT
AATAGTGATGTGGACAAACTATTTATCCAGTTGGTACAAACCTACAA
TCAATTATTTGAAGAAAACCCTATTAACGCAAGTGGAGTAGATGCTA
AAGCGATTCTTTCTGCACGATTGAGTAAATCAAGACGATTAGAAAAT
CTCATTGCTCAGCTCCCCGGTGAGAAGAAAAATGGCTTATTTGGGAA
TCTCATTGCTTTGTCATTGGGTTTGACCCCTAATTTTAAATCAAATT
TTGATTTGGCAGAAGATGCTAAATTACAGCTTTCAAAAGATACTTAC
GATGATGATTTAGATAATTTATTGGCGCAAATTGGAGATCAATATGC
TGATTTGTTTTTGGCAGCTAAGAATTTATCAGATGCTATTTTACTTT
CAGATATCCTAAGAGTAAATACTGAAATAACTAAGGCTCCCCTATCA
GCTTCAATGATTAAACGCTACGATGAACATCATCAAGACTTGACTCT
TTTAAAAGCTTTAGTTCGACAACAACTTCCAGAAAAGTATAAAGAAA
TCTTTTTTGATCAATCAAAAAACGGATATGCAGGTTATATTGATGGG
GGAGCTAGCCAAGAAGAATTTTATAAATTTATCAAACCAATTTTAGA
AAAAATGGATGGTACTGAGGAATTATTGGTGAAACTAAATCGTGAAG
ATTTGCTGCGCAAGCAACGGACCTTTGACAACGGCTCTATTCCCCAT
CAAATTCACTTGGGTGAGCTGCATGCTATTTTGAGAAGACAAGAAGA
CTTTTATCCATTTTTAAAAGACAATCGTGAGAAGATTGAAAAAATCT
TGACTTTTCGAATTCCTTATTATGTTGGTCCATTGGCGCGTGGCAAT
AGTCGTTTTGCATGGATGACTCGGAAGTCTGAAGAAACAATTACCCC
ATGGAATTTTGAAGAAGTTGTCGATAAAGGTGCTTCAGCTCAATCAT
TTATTGAACGCATGACAAACTTTGATAAAAATCTTCCAAATGAAAAA
GTACTACCAAAACATAGTTTGCTTTATGAGTATTTTACGGTTTATAA
CGAATTGACAAAGGTCAAATATGTTACTGAAGGAATGCGAAAACCAG
CATTTCTTTCAGGTGAACAGAAGAAAGCCATTGTTGATTTACTCTTC
AAAACAAATCGAAAAGTAACCGTTAAGCAATTAAAAGAAGATTATTT
CAAAAAAATAGAATGTTTTGATAGTGTTGAAATTTCAGGAGTTGAAG
ATAGATTTAATGCTTCATTAGGTACCTACCATGATTTGCTAAAAATT
ATTAAAGATAAAGATTTTTTGGATAATGAAGAAAATGAAGATATCTT
AGAGGATATTGTTTTAACATTGACCTTATTTGAAGATAGGGAGATGA
TTGAGGAAAGACTTAAAACATATGCTCACCTCTTTGATGATAAGGTG
ATGAAACAGCTTAAACGTCGCCGTTATACTGGTTGGGGACGTTTGTC
TCGAAAATTGATTAATGGTATTAGGGATAAGCAATCTGGCAAAACAA
TATTAGATTTTTTGAAATCAGATGGTTTTGCCAATCGCAATTTTATG
CAGCTGATCCATGATGATAGTTTGACATTTAAAGAAGACATTCAAAA
AGCACAAGTGTCTGGACAAGGCGATAGTTTACATGAACATATTGCAA
ATTTAGCTGGTAGCCCTGCTATTAAAAAAGGTATTTTACAGACTGTA
AAAGTTGTTGATGAATTGGTCAAAGTAATGGGGCGGCATAAGCCAGA
AAATATCGTTATTGAAATGGCACGTGAAAATCAGACAACTCAAAAGG
GCCAGAAAAATTCGCGAGAGCGTATGAAACGAATCGAAGAAGGTATC
AAAGAATTAGGAAGTCAGATTCTTAAAGAGCATCCTGTTGAAAATAC
TCAATTGCAAAATGAAAAGCTCTATCTCTATTATCTCCAAAATGGAA
GAGACATGTATGTGGACCAAGAATTAGATATTAATCGTTTAAGTGAT
TATGATGTCGATCACATTGTTCCACAAAGTTTCCTTAAAGACGATTC
AATAGACAATAAGGTCTTAACGCGTTCTGATAAAAATCGTGGTAAAT
CGGATAACGTTCCAAGTGAAGAAGTAGTCAAAAAGATGAAAAACTAT
TGGAGACAACTTCTAAACGCCAAGTTAATCACTCAACGTAAGTTTGA
TAATTTAACGAAAGCTGAACGTGGAGGTTTGAGTGAACTTGATAAAG
CTGGTTTTATCAAACGCCAATTGGTTGAAACTCGCCAAATCACTAAG
CATGTGGCACAAATTTTGGATAGTCGCATGAATACTAAATACGATGA
AAATGATAAACTTATTCGAGAGGTTAAAGTGATTACCTTAAAATCTA
AATTAGTTTCTGACTTCCGAAAAGATTTCCAATTCTATAAAGTACGT
GAGATTAACAATTACCATCATGCCCATGATGCGTATCTAAATGCCGT
CGTTGGAACTGCTTTGATTAAGAAATATCCAAAACTTGAATCGGAGT
TTGTCTATGGTGATTATAAAGTTTATGATGTTCGTAAAATGATTGCT
AAGTCTGAGCAAGAAATAGGCAAAGCAACCGCAAAATATTTCTTTTA
CTCTAATATCATGAACTTCTTCAAAACAGAAATTACACTTGCAAATG
GAGAGATTCGCAAACGCCCTCTAATCGAAACTAATGGGGAAACTGGA
GAAATTGTCTGGGATAAAGGGCGAGATTTTGCCACAGTGCGCAAAGT
ATTGTCCATGCCCCAAGTCAATATTGTCAAGAAAACAGAAGTACAGA
CAGGCGGATTCTCCAAGGAGTCAATTTTACCAAAAAGAAATTCGGAC
AAGCTTATTGCTCGTAAAAAAGACTGGGATCCAAAAAAATATGGTGG
TTTTGATAGTCCAACGGTAGCTTATTCAGTCCTAGTGGTTGCTAAGG
TGGAAAAAGGGAAATCGAAGAAGTTAAAATCCGTTAAAGAGTTACTA
GGGATCACAATTATGGAAAGAAGTTCCTTTGAAAAAAATCCGATTGA
CTTTTTAGAAGCTAAAGGATATAAGGAAGTTAAAAAAGACTTAATCA
TTAAACTACCTAAATATAGTCTTTTTGAGTTAGAAAACGGTCGTAAA
CGGATGCTGGCTAGTGCCGGAGAATTACAAAAAGGAAATGAGCTGGC
TCTGCCAAGCAAATATGTGAATTTTTTATATTTAGCTAGTCATTATG
AAAAGTTGAAGGGTAGTCCAGAAGATAACGAACAAAAACAATTGTTT
GTGGAGCAGCATAAGCATTATTTAGATGAGATTATTGAGCAAATCAG
TGAATTTTCTAAGCGTGTTATTTTAGCAGATGCCAATTTAGATAAAG
TTCTTAGTGCATATAACAAACATAGAGACAAACCAATACGTGAACAA
GCAGAAAATATTATTCATTTATTTACGTTGACGAATCTTGGAGCTCC
CGCTGCTTTTAAATATTTTGATACAACAATTGATCGTAAACGATATA
CGTCTACAAAAGAAGTTTTAGATGCCACTCTTATCCATCAATCCATC
ACTGGTCTTTATGAAACACGCATTGATTTGAGTCAGCTAGGAGGTGA
CTGAATTAATGCGGCTGCAATTTTTTTGGGCGGGGCCGCCCAAAAAA
ATCCTAGCACCCTGCAGCAGTACTGCTTGACCATAAGAACAAAAAAA
CTTCCGATAAAGTTTGGAAGATAAAGCTAAAAGTTCTTATCTTTGCA
GTTGATGGAGAGGTGCAAGTAGGTTTTAGAGCTAGAAATAGCAAGTT
AAAATAAGGCTAGTCCGTTATCAACTTGAAAAAGTGGCACCGAGTCG
GTGCTTTTTTTGAGATCTGTCGACTCTAGAGGATCCCCGGGTACCGA
GCTCGAATTCACTGGCCGTCGTTTTACAACGTCGTGACTGGGAAAAC
CCTGGCGTTACCCAACTTAATCGTACTTGTGCCTGTTCTATTTCCGA
ACCGACCGCTTGTATGAATCCATCAAAATTCGTTTTCTCTATGTTGG
ATTCCTTGTTGCTCATATTGTGATGATAATTTCTACAAATATAGTCA
TTGGTAACTATCTATGAAACTGTTTGATACTTTTATAGTTGATTAAA
CTTGTTCATGGCATTTGCCTTAATATCATCCGCTATGTCAATGTAGG
GTTTCATAGCTTTGTAGTCGCTGTGTCCCGTCCATTTCATGACCACC
TGTGCCGGGATTCCGAGAGCCAGCGCATTGCAGATGAATGTCCTTCT
TCCTGCATGGGTACTGAGCAAAGCGTATTTGGGTGTGACTTCATCAA
TACGTTCATTTCCCTTGTAGTAGGTTTCCCGTACAGGCTCGTTGATT
TCTGCCAGTTCGCCCAGCTCTTTCAGGTAATCGTTCATCTTCTGGTT
GCTGATGACGGGCAGAGCCATGTAATTCTCGAAATGGATGTCCTTGT
ATTTGTCCAGTATGGCTTTGCTGTATTTGTTCAGTTCAATCGTCAGG
CTGTCGGCAGTCTTGACTGTGGTTATTTCGATGTGGTCGGACTTCAC
ATCGCTTCTTTTCAGATTGCGAACATCCGAATACCGCAAACTCGTAA
AGCAGCAGAACAGGAAAACATCACGCACACGTTCCAGGTATTGCTTA
TCCTTGGGTATCTGGTAGTCTTTCAGCTTGTTCAGTTCATCCCAAGT
CAGGAAGATTACTTTTTTCGAGGTGGTTTTCAGTTTCGGTTTGAACG
TATCGTATGCAATGTTCTGATGATGTCCTTTCTTGAAGCTCCAGCGC
AGGAACCATTTGAGGAATCCCATTTGCTTGCCGATGGTGCTGTTTCT
CATATCCTTGGTGTCACGCAGGAAGTTGACGTATTCGTTCAATCCAA
ACTCGTTGAAATAGTTGAACGTTGCATCCTCCTTGAACTCTTTGAGG
TGGTTCCTCACTGCTGCAAATTTTTCATAGGTGGATGCCGTCCAGTT
ATTCTGGTTACCGCACTCTTTTACAAACTCATCGAACACCTCCCAAA
AGCTGACAGGGGCTTCTTCCGGCTGTTCTTCGCTGGTGTCTTTCATT
CTCATGTTGAAAGCTTCCTTCAACTGTTGGGTCGTTGGCATGACCTC
CTGCACCTCAAATTCCTTGAAAATATTCTGGATTTCGGCATAGTATT
TCAGCAAGTCCGTATTGATTTCGGCTGCACTTTGCTTTAGCTTGTTG
GTACATCCGCTCTTTACCCGCTGCTTATCTGCATCCCATTTGGCTAC
GTCAATCCGGTAGCCCGTTGTAAACTCGATGCGTTGGCTGGCAAAGA
TGACACGCATACGGATGGGTACGTTCTCTACGATTGGCACACCGTTC
TTTTTCCGGCTCTCCAATGCAAAAATGATGTTGCGCTTGATATTCAT
AATTGGGTGCGTTTGAAATTCTACACCCAAATATACACCCAATTATT
GAGATAGCAAAAGACATTTAGAAACATTTACTTTTACTCTATATTGT
AATTTACACTTGATTATCAGTCGTTTGCAGTCTTATGATATTCTGTG
AAAGTATAAGTTCGAGAGCCTGTCTCTCCGCAAAAAACGCTGAAAAT
CAGCAGATTGCAAAACAAACACCCTGTTTTACACCCAAGAATGTAAA
GTCGGCTGTTTTTGTTTTATTTAAGATAATACAACCACTACATAATA
AAAGAGTAGCGATATTAAAAGAATCCGATGAGAAAAGACTAATATTT
ATCTATCCATTCAGTTTGATTTTTCAGGACTTTACATCGTCCTGAAA
GTATTTGTTGGTACCGGTACCGAGGACGCGTAAACATTTACAGTTGC
ATGTGGCCTATTGTTTTTAAGATTTTACTGAAGTTTGCATTAATAAA
GAATATACTACAGCTGATATATGCGCAACATATTGTGACGCTTGTGA
TTTATTTCCCTTGAAATCCTTAACAAATACCGCTAAGGTATAACTGA
TATTATTAGGCAGACATATATAGGCAACATCATTGTGAGCTGCAAGA
ACACCATTTTCATTAACATAACCTGAACCTGTCTTATGCGCTATAAC
AACCCCTTCTTTATCAAGAAGTGGAGCTGCTATCCTATCTACACCTG
TTTTGCATTCTTTTAACGTATTCTTAATGAAACTTTGTTTCTCATCA
TCGATAAGACCTTCAGTAAACAAACGATTCATCAACATTGCAGCACC
AAGAGGAGATGTATAGTTAGAGTAAGCCTTGTTATGGTCAGCCGACA
TTTCCTCTTCCGTATAAGCTATCTGAAAACTTGAACGAGGAATGAGT
GTGGCTATAAAACTATCTGTTTGAGCGACATTAACCATATCCTTAAA
CATAAGGTTGCTTGCATTGTTGTCACTCTGAGTAAGAGTATAACGCA
GCAAATCTCTCACTGTCAATGATATGACTGGCCCTGAATAATCTTTC
AGCATAGGACTCCAAGTCTTTGGGTCAAGTTTATCCCTATTTATATT
TACTAAGGTATCAAGTGAAATTCCTTTATTGTCAAAGTCATTACAAA
GAGCTAATGCCTGATGAACCTTAAACACACTCATCATAGGATAAACA
CTCTTATTATTGACCTTAACCGTATCTCTGTTATTAACAATAACCGC
CACACCAATTTCGCCAGGACAAGCTGAGACAATTTGAGAAATGCTAT
CAGTCAAAACATTTGTTAAAGGAGGATTTGCGCTATCTTTTGTCGCT
GATTTATGGAACAATGAAAATACCAAGATGAAAATGCAAACTAAAGC
TATACTCAAAACTACGATTTGTTTTTTTCTGTTTTTTTCCATTATAA
CCTCTCCTTAATTTATTGCATCTCTTTTCGAATATTTATGTTTTTTG
AGAAAAGAACGTACTCATGGTTCATCCCGATATGCGTATCGGTCTGT
ATATCAGCAACTTTCTATGTGTTTCAACTACAATAGTCATCTATTCT
CATCTTTCTGAGTCCACCCCCTGCAAAGCCCCTCTTTACGACATAAA
AATTCGGTCGGAAAAGGTATGCAAAAGATGTTTCTCTCTTTAAGAGA
AACTCTTCGGGATGCAAAAATATGAAAATAACTCCAATTCACCAAAT
TATATAGCGACTTTTTTACAAAATGCTAAAATTTGTTGATTTCCGTC
AAGCAATTGTTGAGCAAAAATGTCTTTTACGATAAAATGATACCTCA
ATATCAACTGTTTAGCAAAACGATATTTCTCTTAAAGAGAGAAACAC
CTTTTTGTTCACCAATCCCCGACTTTTAATCCCGCGGCCATGATTGA
AAAAGGAAGAGTATGAGTATTCAACATTTCCGTGTCGCCCTTATTCC
CTTTTTTGCGGCATTTTGCCTTCCTGTTTTTGCTCACCCAGAAACGC
TGGTGAAAGTAAAAGATGCTGAAGATCAGTTGGGTGCACGAGTGGGT
TACATCGAACTGGATCTCAACAGCGGTAAGATCCTTGAGAGTTTTCG
CCCCGAAGAACGTTTTCCAATGATGAGCACTTTTAAAGTTCTGCTAT
GTGGCGCGGTATTATCCCGTATTGACGCCGGGCAAGAGCAACTCGGT
CGCCGCATACACTATTCTCAGAATGACTTGGTTGAGTACTCACCAGT
CACAGAAAAGCATCTTACGGATGGCATGACAGTAAGAGAATTATGCA
GTGCTGCCATAACCATGAGTGATAACACTGCGGCCAACTTACTTCTG
ACAACGATCGGAGGACCGAAGGAGCTAACCGCTTTTTTGCACAACAT
GGGGGATCATGTAACTCGCCTTGATCGTTGGGAACCGGAGCTGAATG
AAGCCATACCAAACGACGAGCGTGACACCACGATGCCTGTAGCAATG
GCAACAACGTTGCGCAAACTATTAACTGGCGAACTACTTACTCTAGC
TTCCCGGCAACAATTAATAGACTGGATGGAGGCGGATAAAGTTGCAG
GACCACTTCTGCGCTCGGCCCTTCCGGCTGGCTGGTTTATTGCTGAT
AAATCTGGAGCCGGTGAGCGTGGGTCTCGCGGTATCATTGCAGCACG
GGGCCAGATGGTAAGCCCTCCCGTATCGTAGTTATCTACACGACGGG
GAGTCAGGCAACTATGGATGAACGAAATAGACAGATCGCTGAGATAG
GTGCCTCACTGATTAAGCATTGGTAACTGTCAGACCAAGTTTACTCA
TAACGCGTCAATTCGAGGGGGATCAATTCCGTGATAGGTGGGCTGCC
CTTCCTGGTTGGCTTGGTTTCATCAGCCATCCGCTTGCCCTCATCTG
TTACGCCGGCGGTAGCCGGCCAGCCTCGCAGAGCAGGATTCCCGTTG
AGCACCGCCAGGTGCGAATAAGGGACAGTGAAGAAGGAACACCCGCT
CGCGGGTGGGCCTACTTCACCTATCCTGCCCGGCTGACGCCGTTGGA
TACACCAAGGAAAGTCTACACGAACCCTTTGGCAAAATCCTGTATAT
CGTGCGAAAAAGGATGGATATACCGAAAAAATCGCTATAATGACCCC
GAAGCAGGGTTATGCAGCGGAAAACGGAATTGATCCGGCCACGATGC
GTCCGGCGTAGAGGATCTGAAGATCAGCAGTTCAACCTGTTGATAGT
ACGTACTAAGCTCTCATGTTTCACGTACTAAGCTCTCATGTTTAACG
TACTAAGCTCTCATGTTTAACGAACTAAACCCTCATGGCTAACGTAC
TAAGCTCTCATGGCTAACGTACTAAGCTCTCATGTTTCACGTACTAA
GCTCTCATGTTTGAACAATAAAATTAATATAAATCAGCAACTTAAAT
AGCCTCTAAGGTTTTAAGTTTTATAAGAAAAAAAAGAATATATAAGG
CTTTTAAAGCTTTTAAGGTTTAACGGTTGTGGACAACAAGCCAGGGA
TGTAACGCACTGAGAAGCCCTTAGAGCCTCTCAAAGCAATTTTGAGT
GACACAGGAACACTTAACGGCTGACATGGGAATTCCCCTCCACCGCG
GTGG

### Example 14

### CRISPR Integration on the Chromosome of Bacteroides cellulosilyticus WH2

The pNBU2-CRISPR plasm ids were transformed to E. coli S-17 lambda-pir, followed by delivery to Bacteroides cellulosilyticus WH2 cells via conjugation. In this specific example, the pNBU2-CRISPR plasmid encodes the intN2 tyrosine integrase, which mediates sequence-specific recombination between the attN2 site on pNBU2-CRISPR plasmid and one of three attBWH2 sites located in the 3′ ends of the two tRNA-Ser genes, BcellWH2_RS22795 or BcellWH2_RS23000, or a non-coding region (nucleotide coordinates 6,071,791-6,071,803) on the chromosome of Bacteroides cellulosilyticus WH2 (BWH2 in short). Insertion of the pNBU2-CRISPR plasmid may inactivate one of the two tRNA-Ser genes (will not inactivate a tRNA-Ser gene if inserted in the non-coding region), and simultaneous insertion into both tRNA-Ser genes is unlikely because of the essentiality of tRNA-Ser.

Five plasmids were constructed which express a non-targeting control guide (termed ‘M’), two guide RNAs targeting tdk_BWH2 (BcellWH2_RS17975) (termed ‘T2’ and ‘T3’) and two guide RNAs targeting susC_BWH2 (BcellWH2_RS26295) (termed ‘S6’ and ‘S19’). The tdk gene encodes thymidine kinase, and the susC gene encodes the SusC/RagA family Ton-B-linked outer membrane protein involved in starch binding in Bacteroides cellulosilyticus WH2. The two protospacer sequences for tdk_BWH2 are T2 (5′-ATACAGGAAACCAATCGTAG-3′; SEQ ID NO:11) and T3, (5′-GGAAGAATCGAAGTTATATG-3′; SEQ ID NO:12) and for susC_BWH2 are S6 (5′-AATCCACTGGATGCCATCCG-3′; SEQ ID NO:13) and S19 (5′-GCTTATGTCTATCTATCCGG-3′; SEQ ID NO:14). In silico analysis of the non-targeting control protospacer sequence M (5′-TGATGGAGAGGTGCAAGTAG-3′; SEQ ID NO 6) against Bacteroides genomes did not result in any significant sequence matches, so no “off-target” activity is expected. The sgRNA scaffold sequence was 5′-GTTTTAGAGCTAGAAATAGCAAGTTAAAATAAGGCTAGTCCGTTATCAA CTTGAAAAAGTGGCACCGAGTCGGTGCTTTTTT-3′ (SEQ ID NO:7). The resulting plasmids are called pNBU2-CRISPR.M_BWH2, pNBU2-CRISPR.tdk_BWH2-2, pNBU2-CRISPR.tdk_BWH2-3, pNBU2-CRISPR.susC_BWH2-6 and pNBU2-CRISPR.susC_BWH2-19.

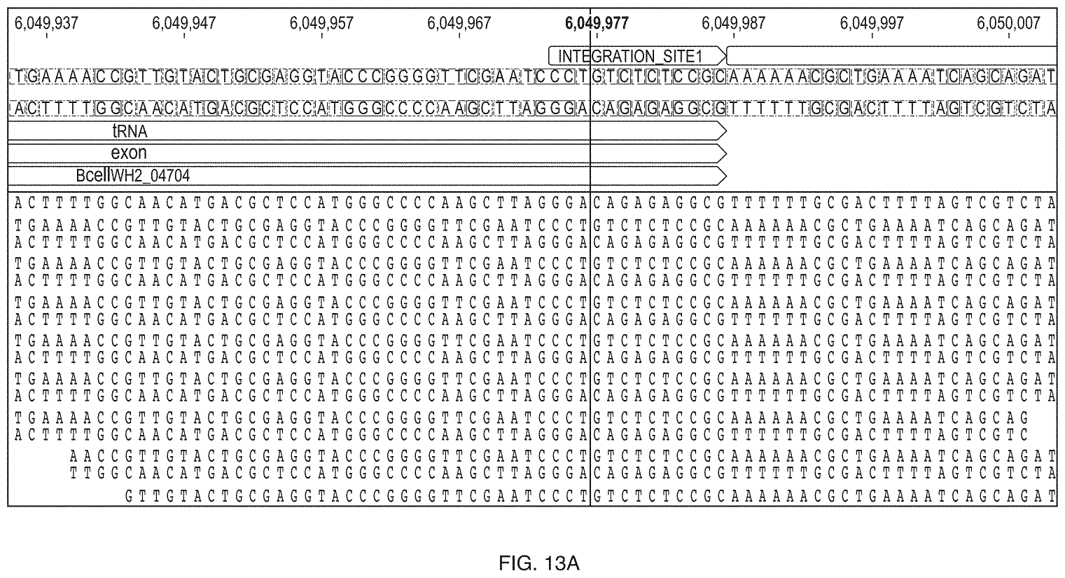

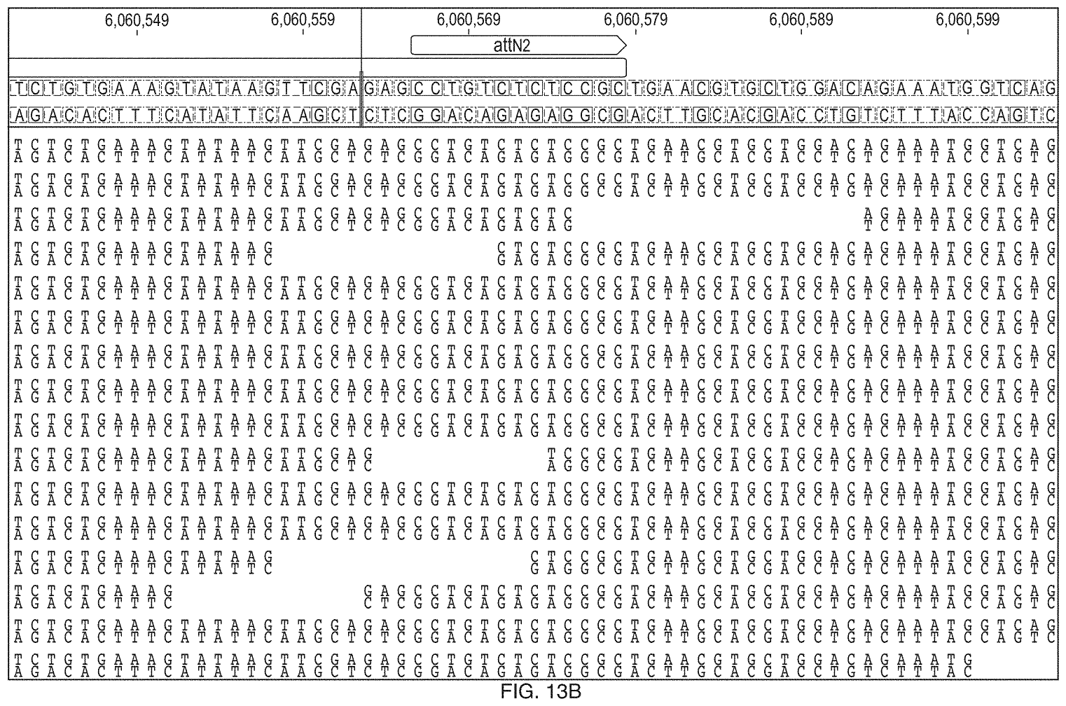

An example of a targeted insertion between the attN2 site on a pNBU2-CRISPR plasmid with cefoxitin resistance and one of three attBWH2 sites in the Bacteroides cellulosilyticus WH2 genome is shown in FIGS. 13A-B. Plasmid pNBU2-CRISPR.susC_BWH2-19 integrates only in the attBWH2 site in the t-RNA-Ser gene, BcellWH2_RS22795. The 5′ end of the plasmid integration site is shown in FIG. 13A and the 3′ end of the plasmid integration site is shown in FIG. 13B. Sequencing was performed using IIlumina Next Gen Sequencing technology, and the analysis was done with Geneious align/assemble software.

### Example 15

### Inducible CRISPR Killing of Individual B. cellulosilyticus WH2 Strains

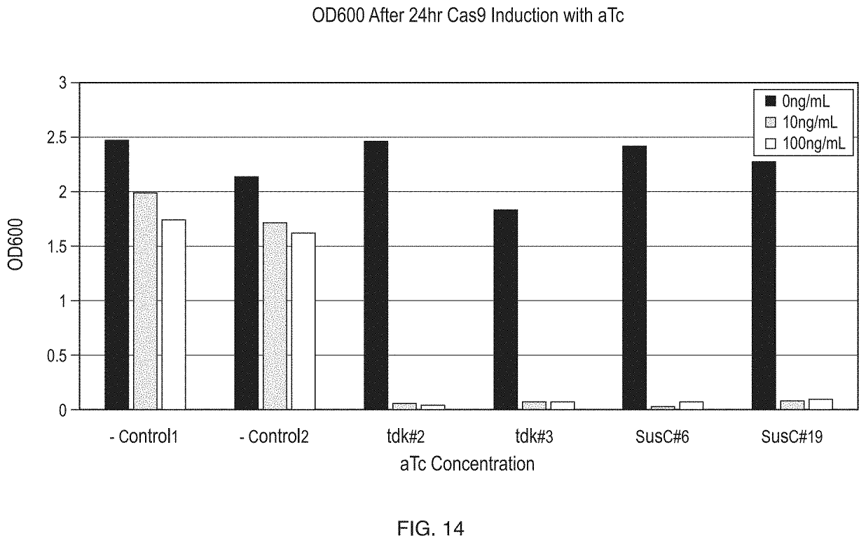

For selected Bacteroides cellulosilyticus WH2 integrants (M1, M2, T2, T3, S6 and S19), inducible CRISPR Cas9 mediated cell killing was investigated in TYG liquid medium. Single colonies of M1, M2, T2, T3, S6 and S19 (M1 and M2 were separate colonies from the same M non-targeting conjugation plate) were grown anaerobically in a coy chamber (coy Laboratory Products Inc.) overnight in falcon tubes cultures containing 5 ml of TYG liquid medium supplemented with 200 μg/ml gentamicin (Gm) and 10 μg/ml cefoxitin (FOX). Overnight cultures were then normalized to an OD_{600 nm} of 1 with TYG medium. These normalized cultures were diluted at a 1:100 ratio (250 μl into 24.75 ml of TYG) to make a seed culture. This 25 ml seed culture was aliquoted into 5 ml cultures in fresh 15 ml falcon tubes. Anhydrotetracycline (aTc) was added to the 5 ml cultures at either 0 ng/ml. 10 ng/ml or 100 ng/ml and incubated anaerobically at 37° C. for 24 hours. OD_{600 nm} readings taken after 24 hours of growth are shown in FIG. 14. The data shows a chromosomally integrated CRISPR/Cas9 system is activated by an exogenously provided inducer (aTc) to generate lethal genomic DNA cleavage guided by a targeting RNA (tdk_BWH2 or susC_BWH2), resulting in loss of cell viability.

### REFERENCES

Goodman, A. L. et al. Identifying genetic determinants needed to establish a human gut symbiont in its habitat. Cell Host Microbe 6, 279-289 (2009).

Hibberd, M. C. et al. The effects of micronutrient deficiencies on bacterial species from the human gut microbiota. Sci. Transl. Med. 9, eaal4069 (2017).

Patnode, M. et al. Interspecies competition impacts targeted manipulation of human gut bacteria by fiber-derived glycans. Cell, 179(1), 59-73.

Lenth, R. (2019). emmeans: Estimated Marginal Means, aka Least-Squares Means. R package version 1.4.1. https://CRAN.R-project.org/package=emmeans.

Ridaura, V. K. et al. Gut microbiota from twins discordant for obesity modulate metabolism in mice. Science 341, 1241214.

R Core Team (2018). R: A language and environment for statistical computing. R Foundation for Statistical Computing, Vienna, Austria. URL https://www.R-project.org/.

Smith, C. J. et al. Nucleotide Sequence Determination and Genetic Analysis of the Bacteroides Plasmid, pBI143. Plasmid, 1995, 34:211-222

Lim et al. Engineered Regulatory Systems Modulate Gene Expression of Human Commensals in the Gut. Cell, 2017, 169:547-558

What is claimed is:

1. A protein-nucleic acid complex comprising an engineered RNA-guided nuclease system in association with a chromosome of a bacterial or archaeal species, wherein the engineered RNA-guided nuclease system is targeted to a site in the chromosome of the microorganism, and the chromosome of the microorganism encodes an HU family DNA-binding protein comprising an amino acid sequence with at least 50% sequence identity to SEQ ID NO:1.

2. The protein-nucleic acid complex of claim 1, wherein the engineered RNA-guided nuclease system is a CRISPR system chosen from a Type I CRISPR system, a type II CRISPR system, a type III CRISPR system, a Type IV CRISPR system, a type V CRISPR system, or a type VI CRISPR system.

3. The protein-nucleic acid complex of claim 2, wherein the CRISPR system comprises a CRISPR nuclease and a guide RNA.

4. The protein-nucleic acid complex of claim 3, wherein the CRISPR nuclease is Cas9, Cas12, Cas13, or CasX.

5. The protein-nucleic acid complex of claim 1, wherein the engineered RNA-guided nuclease system is expressed from a nucleic acid that encodes the engineered RNA-guided nuclease system and is integrated into the bacterial or archaeal chromosome.

6. The protein-nucleic acid complex of claim 1, wherein the engineered RNA-guided nuclease system is expressed from a nucleic acid that encodes the engineered RNA-guided nuclease system and is carried on an extrachromosomal vector.

7. The protein-nucleic acid complex of claim 1, wherein the prokaryotic chromosome is within a Bacteroides species.

8. The protein-nucleic acid complex of claim 7, wherein the Bacteroides species is chosen from B. thetaiotaomicron, B. vulgatus, B. cellulosilyticus, B. fragilis, B. helcogenes, B. ovatus, B. salanitronis, B. uniformis, or B. xylanisolvens.

9. A method for slowing growth of a target prokaryote in a mixed population of prokaryotes, the method comprising expressing an engineered RNA-guided nuclease system in the target prokaryote, wherein the engineered RNA-guided nuclease system is targeted to a site in a chromosome of the target prokaryote such that at least one double stranded break is introduced in the chromosome of the target prokaryote, thereby slowing growth of the target prokaryote.

10. The method of claim 9, wherein slowing growth of the target prokaryote leads to reduced or eliminated levels of the target prokaryote in the mixed population of prokaryotes.

11. The method of claim 9, wherein expression of the RNA-guided nuclease system is inducible.

12. The method of claim 9, wherein the engineered RNA-guided nuclease system is a CRISPR system chosen from a Type I CRISPR system, a type II CRISPR system, a type III CRISPR system, a Type IV CRISPR system, a type V CRISPR system, or a type VI CRISPR system.

13. The method of claim 12, wherein the CRISPR system comprises a CRISPR nuclease and a guide RNA.

14. The method of claim 13, wherein the CRISPR nuclease is a Cas9, a Cas12, a Cas13, or a CasX nuclease.

15. The method of claim 13, wherein the CRISPR nuclease and guide RNA are expressed from at least one nucleic acid integrated into the chromosome of the target prokaryote.

16. The method of claim 13, wherein the CRISPR nuclease and guide RNA are expressed from at least one nucleic acid carried on an extrachromosomal vector.

17. The method of claim 15, wherein the nucleic acid encoding the CRISPR nuclease is operably linked to an inducible promoter.

18. The method of claim 17, wherein the expressing step comprises contacting the mixed population of prokaryotes with a promoter inducing chemical.

19. The method of claim 9, wherein the mixed population of prokaryotes is harbored in cell culture.

20. The method of claim 9, wherein the mixed population of prokaryotes is harbored in a mammal's digestive tract.

21. The method of claim 20, wherein the engineered RNA-guided nuclease system is an engineered CRISPR nuclease system, at least one nucleic acid encoding the engineered CRISPR nuclease system is introduced into the target prokaryote, the nucleic acid encoding the CRISPR nuclease is operably linked to an inducible promoter, and the expressing step comprises administering a promoter inducing chemical to the mammal.

22. The method of claim 21, wherein the administering comprises orally administering the promoter inducing chemical.

23. The method of claim 18, wherein the promoter inducing chemical is anhydrotetracycline.

24. The method of claim 20, wherein the mammal is a human.

25. The method of claim 24, wherein the human is undergoing treatment for cancer, and reduction or elimination of the target prokaryote from the mixed population of prokaryotes in the gastrointestinal tract of the human improves the response of the human to the treatment for cancer.

26. The method of claim 25, wherein the treatment for cancer comprises immunotherapy.

27. The method of claim 9, wherein the target prokaryote is a Bacteroides species.

28. The method of claim 27, wherein the Bacteroides species is chosen from B. thetaiotaomicron, B. vulgatus, B. cellulosilyticus, B. fragilis, B. helcogenes, B. ovatus, B. salanitronis, B. uniformis, or B. xylanisolvens.
